(19) United States
(12) Patent Application Publication
(10) Pub. No.: US 2025/0114601 A1
(43) Pub. Date: Apr. 10, 2025

(54) EPILEPSY THERAPY USING SPINAL CORD STIMULATION

(71) Applicant: Boston Scientific Neuromodulation Corporation, Valencia, CA (US)

(72) Inventors: Jianwen Gu, Richmond, TX (US); Rosana Esteller, Santa Clarita, CA (US)

(21) Appl. No.: 18/893,108

(22) Filed: Sep. 23, 2024

Related U.S. Application Data
(60) Provisional application No. 63/542,683, filed on Oct. 5, 2023.

(51) Int. Cl. A61N 1/36

(52) U.S. Cl. CPC A61N 1/36064; A61N 1/36062; A61N 1/36139; A61N 1/36146

(57) ABSTRACT

A system may be configured for treating a patient with epileptic seizures. The system may include a therapy delivery system configured to use a set of electrodes positioned adjacent to a spinal cord or dorsal column nuclei and a stimulation configuration to deliver neuromodulation energy to at least one target neural pathway in or near the spinal cord. The at least one target neural pathway includes the dorsal column nuclei or axons projecting to the dorsal column nuclei. The dorsal column nuclei and the axons are somatotopically organized and project to at least one of a cerebellum, a thalamus or a brainstem, which include somatotopically organized nuclei and project axons to different areas of a cortex. The at least one target neural pathway corresponds to at least one of the different areas of the cortex in which the epileptic seizures were located.

### CLAIM OF PRIORITY

This application claims the benefit of U.S. Provisional Application No. 63/542,683, filed on Oct. 5, 2023, which is hereby incorporated by reference in its entirety.

### TECHNICAL FIELD

This document relates generally to medical systems, and more particularly, but not by way of limitation, to systems, devices, and methods for providing epilepsy therapy.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators). Implantable stimulation devices may deliver electrical stimuli to treat various biological disorders, such spinal cord stimulators (SCS) to treat chronic pain, cortical and Deep Brain Stimulators (DBS) to treat motor and psychological disorders, and Peripheral Nerve Stimulation (PNS). An example of a PNS system is a vagal nerve stimulation (VNS) system. VNS may be delivered using a cervical vagal nerve implant. Another example of a PNS system is the trigeminal nerve stimulation (TNS) system.

Epilepsy continues to be a difficult condition to treat, For example, different types of stimulation such as VNS, DBS and TNS, have been proposed to treat epilepsy but these proposals typically involve invasive procedures and limited targets for treating epileptic conditions.

### SUMMARY

An example (e.g., Example 1) of a system for treating a patient with epileptic seizures may include a therapy delivery system configured to use a set of electrodes positioned adjacent to a spinal cord or dorsal column nuclei and a stimulation configuration to deliver neuromodulation energy to at least one neural target in or near the spinal cord. The at least one neural target includes the dorsal column nuclei or axons projecting to the dorsal column nuclei. The dorsal column nuclei and the axons are somatotopically organized and the dorsal column nuclei project to at least one of another brainstem area (the dorsal column nuclei are part of the medulla that is part of the brainstem), a cerebellum or a thalamus, and the at least one of the another brainstem area, the cerebellum or the thalamus includes somatotopically organized nuclei and project axons to different areas of at least one of a cortex or a limbic system. The at least one neural target corresponds to at least one of the cortex, the limbic system, the another brainstem area, the cerebellum or the thalamus in which the epileptic seizures propagate.

In Example 2, the subject matter of Example 1 may optionally be configured such that the at least one neural target includes a spinothalamic tract that provides afferent fibers to an intralaminar thalamic nuclei.

In Example 3, the subject matter of any one or more of Examples 1-2 may optionally be configured such that the at least one neural target is based on a symptom location where the patient experiences a symptom caused by the epileptic seizures.

In Example 4, the subject matter of any one or more of Examples 1-3 may optionally be configured such that the epileptic seizures include a first symptom at a first location and a second symptom at a second location, and a first target is based on the first location where the patient experiences the first symptom and a second target is based on the second location where the patient experiences the second symptom.

In Example 5, the subject matter of any one or more of Examples 1-4 may optionally be configured such that the stimulation configuration includes a plurality of pulses in a pulse pattern. The pulse pattern may include at least one of different pulse amplitudes, different pulse widths, different pulse-to-pulse spacing, different pulse shapes, different bursts of pulses, different electrode contacts in the set of electrodes, or different fractionalizations. It is noted that the pattern may be repeated (e.g., periodically delivered pattern).

In Example 6, the subject matter of any one or more of Examples 1-5 may optionally be configured such that the epileptic seizures propagate to two or more areas, and the therapy delivery system is configured to use two or more stimulation configurations corresponding to the two or more areas. The two or more stimulation configurations may include different stimulation patterns, and at least one of the different stimulation patterns may include a stimulation pattern with different pulse amplitudes, different pulse widths, or different pulse-to-pulse spacing.

In Example 7, the subject matter of any one or more of Examples 1-6 may optionally be configured such that the at least one neural target includes axons in at least a portion of a gracile fasciculus or a cuneate fasciculus.

In Example 8, the subject matter of any one or more of Examples 1-7 may optionally be configured such that the at least one neural target includes pathways in at least a portion of a posterolateral tract, lateral spinothalamic tract, an anterior spinothalamatic tract, a spinoreticular tract, a posterior spinocerebellar tract, an anterior spinocerebellar tract, or a spinoolivary tract.

In Example 9, the subject matter of any one or more of Examples 1-8 may optionally be configured such that the at least one neural target includes pathways in at least a portion of trigeminothalamatic tract via a trigeminocervical nucleus.

In Example 10, the subject matter of any one or more of Examples 1-9 may optionally be configured such that the therapy delivery system includes at least one lead in a dorsal epidural space to position at least some of the set of electrodes in the dorsal epidural space.

In Example 11, the subject matter of any one or more of Examples 1-10 may optionally be configured such that the therapy delivery system includes at least one lead fed through a dorsal epidural space and at least partially around the spinal cord to position at least some of the set of electrodes in a lateral or antero-lateral epidural space surrounding the spinal cord.

In Example 12, the subject matter of Examples 11 may optionally be configured such that the stimulation configuration is configured to focus a neuromodulation field to stimulate neural pathways in a portion of a nerve tract without stimulating other neural pathways in other portions of the nerve tract.

In Example 13, the subject matter of any one or more of Examples 1-12 may optionally be configured such that the therapy delivery system is configured to use a first channel to deliver first neuromodulation energy using a first stimulation configuration to provide a first neuromodulation field and use a second channel to deliver second neuromodulation energy using a second stimulation configuration to provide a second neuromodulation field, and to coordinate delivery of the first neuromodulation energy and the second neuromodulation energy. The first neuromodulation field may be configured to stimulate a first site and the second neuromodulation field may be configured to stimulate a second site lateral to the first site, the first neuromodulation field may be configured to stimulate a first nerve tract without stimulating a second nerve tract, and the second neuromodulation field may be configured to stimulate the second nerve tract without stimulating the first nerve tract; or the first neuromodulation field is at a first spinal cord level without extending to a second spinal cord level, and the second neuromodulation field is at the second spinal cord level without extending to the first spinal cord level.

In Example 14, the subject matter of any one or more of Examples 1-13 may optionally be configured to further include at least one event detector to detect at least one predefined epileptic event. The therapy delivery system may be configured to respond to the detected at least one predefined epileptic event by delivering the neuromodulation energy. The at least one event detector may be configured to detect the at least one predefined epileptic event using at least one of: received user input; sensed electrical signals in a brain; sensed cardiac activity; sensed blood oxygen; sensed respiration; sensed movement or lack of movement; sensed body temperature; sensed neurotransmitter or biochemical component; sensed sound, sensed ultrasound, or analysis of an image of a patient.

In Example 15, the subject matter of Example 14 may optionally be configured such that the at least one event detector is configured to detect at least a first predefined epileptic event and a second predefined epileptic event. The therapy delivery system may be configured to respond to the first predefined epileptic event by delivering a first neuromodulation therapy and respond to the second predefined epileptic event by delivering a second neuromodulation therapy. The first and second neuromodulation therapies may differ in at least one of a neural target or use different neural stimulation patterns.

Example 16 includes subject matter (such as a method, means for performing acts, machine readable medium including instructions that when performed by a machine cause the machine to perform acts, or an apparatus to perform). The subject matter may be configured for treating a patient with epileptic seizure. The subject matter may include using a set of electrodes epidurally positioned adjacent to a spinal cord or dorsal column nuclei and a stimulation configuration to deliver neuromodulation energy to at least one neural target in or near the spinal cord. The neural target may include the dorsal column nuclei or axons projecting to the dorsal column nuclei. The dorsal column nuclei and the axons are somatotopically organized and the dorsal column nuclei project to at least one of another brainstem area, a cerebellum or a thalamus. The at least one of the another brainstem area, the cerebellum or the thalamus includes somatotopically organized nuclei and project axons to different areas of a cortex or a limbic system. The at least one target neural pathway corresponds to at least one of the cortex, the limbic system, the another brainstem area, the cerebellum or the thalamus propagate-.

In Example 17, the subject matter of Example 16 may optionally be configured such that the at least one neural target pathway includes a spinothalamic tract to provide afferent fibers to an intralaminar thalamic nuclei.

In Example 18, the subject matter of Example 17 may optionally be configured such that the at least one neural target is based on a symptom location where the patient experiences a symptom caused by the epileptic seizures.

In Example 19, the subject matter of any one or more of Examples 16-18 may optionally be configured such that the epileptic seizures include a first symptom at a first location and a second symptom at a second location, and a first target is based on the first location where the patient experiences the first symptom and a second target is based on the second location where the patient experiences the second symptom.

In Example 20, the subject matter of any one or more of Examples 16-19 may optionally be configured such that the stimulation configuration includes a plurality of pulses in a pulse pattern. The pulse pattern may include at least one of different pulse amplitudes, different pulse widths, different pulse-to-pulse spacing, different pulse shapes or different bursts of pulses, different electrode contacts in the set of electrodes, or different fractionalizations. It is noted that the pattern may be repeated (e.g., a periodically delivered pattern).

In Example 21, the subject matter of any one or more of Examples 16-20 may optionally be configured such that the epileptic seizures propagate in two or more areas. The subject matter is configured to use two or more stimulation configurations corresponding to the two or more areas. The two or more stimulation configurations may include different stimulation patterns, and at least one of the different stimulation patterns may include a stimulation pattern with different pulse amplitudes, different pulse widths, or different pulse-to-pulse spacing.

In Example 22, the subject matter of any one or more of Examples 16-21 may optionally be configured such that the at least one neural target includes axons in at least a portion of a gracile fasciculus or a cuneate fasciculus.

In Example 23, the subject matter of any one or more of Examples 16-22 may optionally be configured such that the at least one neural target includes pathways in at least a portion of a posterolateral tract, lateral spinothalamic tract, an anterior spinothalamatic tract, a spinoreticular tract, a posterior spinocerebellar tract, an anterior spinocerebellar tract, or a spinoolivary tract.

In Example 24, the subject matter of any one or more of Examples 16-23 may optionally be configured such that the at least one neural target includes pathways in at least a portion of trigeminothalamatic tract via a trigeminocervical nucleus.

In Example 25, the subject matter of any one or more of Examples 16-24 may optionally be configured to use at least one lead in a dorsal epidural space to position at least some of the set of electrodes in the dorsal epidural space.

In Example 26, the subject matter of any one or more of Examples 16-25 may optionally be configured to use at least one lead fed through a dorsal epidural space and at least partially around the spinal cord to position at least some of the set of electrodes in a lateral or antero-lateral epidural space surrounding the spinal cord.

In Example 27, the subject matter of any one or more of Examples 16-26 may optionally be configured such that the stimulation configuration is configured to focus a neuromodulation field to stimulate neural pathways in a portion of a nerve tract without stimulating other neural pathways in other portions of the nerve tract.

In Example 28, the subject matter of any one or more of Examples 16-27 may optionally be configured to include using a first channel and a first stimulation configuration to deliver first neuromodulation energy to provide a first neuromodulation field, using a second channel and a second stimulation configuration to deliver second neuromodulation energy to provide a second neuromodulation field, and coordinating delivery of the first neuromodulation energy and the second neuromodulation energy. The first neuromodulation field may be configured to stimulate a first site and the second neuromodulation field may be configured to stimulate a second site lateral to the first site; the first neuromodulation field may be configured to stimulate a first nerve tract without stimulating a second nerve tract, and the second neuromodulation field may be configured to stimulate the second nerve tract without stimulating the first nerve tract; or the first neuromodulation field may be at a first spinal cord level without extending to a second spinal cord level, and the second neuromodulation field may be at the second spinal cord level without extending to the first spinal cord level.

In Example 29, the subject matter of any one or more of Examples 16-28 may optionally be configured to further include using at least one event detector to detect at least one predefined epileptic event and responding to the detected at least one predefined epileptic event by delivering the neuromodulation energy.

In Example 30, the subject matter of Example 29 may optionally be configured such that the at least one event detector is configured to detect at least a first predefined epileptic event and a second predefined epileptic event. The method may include responding to the first predefined epileptic event by delivering a first neuromodulation therapy and responding to the second predefined epileptic event by delivering a second neuromodulation therapy. The first and second neuromodulation therapies may differ in at least one of a neural target or use different neural stimulation patterns.

Example 31 includes subject matter (such as a method, means for performing acts, machine readable medium including instructions that when performed by a machine cause the machine to perform acts, or an apparatus to perform). The subject matter may include delivering at least one epilepsy therapy to at least one therapy target in or near a spinal cord using a set of electrodes to provide therapy data. The therapy data may include therapy configuration data. The at least one therapy target is used to transmit impulses to at least one of a cerebellum, a thalamus, a brainstem or a limbic system. The subject matter may further include detecting one or more epileptic events to compile event data, providing condition data indicative of an effect that the delivered at least one therapy has on a treated condition, and analyzing the event data, the therapy configuration data, and the condition data to determine whether the at least one therapy is effective in treating the one or more epileptic events when delivered in response to the detected one or more epileptic events, and to define one or more event-therapy relationships associating the at least one epilepsy therapy to be delivered in response to the one or more detected epileptic events.

In Example 32, the subject matter of Example 31 may optionally be configured such that the delivered at least one epilepsy therapy includes a waveform pattern, an active subset of the electrodes, and fractionalized energy electrode contributions using the active subset of the electrodes. Analyzing the event data, the therapy configuration data, and the condition data includes determining whether the waveform patterns and the fractionalized energy electrode contributions are effective. The subject matter may further include using machine learning to adjust therapy parameters including at least one of the waveform pattern, the active subset of the electrodes, or fractionalized energy electrode contributions using the active subset of the electrodes based on the determined effectiveness until the adjusted therapy parameters are effective.

In Example 33, the subject matter of Example 32 may optionally be configured such that the fractionalized electrode contributions provide a modulation field to modulate only a subset of neural pathways within a nerve tract without modulating other neural pathways within the nerve tract.

In Example 34, the subject matter of any one or more of Examples 31-33 may optionally be configured such that the detected one or more epileptic events includes at least a first stage and a second stage for progression of an epileptic condition. The defined one or more event-therapy relationships may associate the one or more of the least two therapies to be delivered for at least the first stage and the second stage.

In Example 35 the subject matter of any one or more of Examples 31-34 may optionally be configured such that the detected one or more epileptic events includes a detected seizure event and a detected end to the seizure event. The defined one or more event-therapy relationships may associate the one or more of the least two therapies to be delivered for at least the seizure event and the end to the seizure event.

An example (e.g., Example 36) of a system may be configured for treating a patient with epileptic seizures. The system may include a therapy delivery system configured to use a set of electrodes positioned adjacent to a spinothalamic tract that provides afferent fibers to the intralaminar nuclei.

An example (e.g., Example 37) of a system for treating a patient with epileptic seizures may include a therapy delivery system configured to use a set of electrodes positioned to deliver neuromodulation energy to at least one target neural pathway in or near the spinal cord. The at least one target neural pathway is based on a symptom location where the patient experiences a symptom of the epileptic seizures.

An example (e.g., Example 38) of a system for treating a patient with epileptic seizures may include a therapy delivery system configured to use a set of electrodes positioned to deliver neuromodulation energy to at least one target neural pathway in or near the spinal cord and a stimulation configuration that includes a plurality of pulses in a pulse pattern, wherein the pulse pattern includes at least one of different pulse amplitudes, different pulse widths, different pulse-to-pulse spacing, different pulse shapes or different bursts of pulses.

An example (e.g., Example 39) of a system for treating a patient with epileptic seizures may include at least one lead fed through a dorsal epidural space and at least partially around the spinal cord to position at least some of the set of electrodes lateral to the spinal cord.

An example (e.g., Example 39) of a system for treating a patient with epileptic seizures may optionally be configured such that the therapy delivery system is configured to use a first channel to deliver first neuromodulation energy using a first stimulation configuration to provide a first neuromodulation field and use a second channel to deliver second neuromodulation energy using a second stimulation configuration to provide a second neuromodulation field, and to coordinate delivery of the first neuromodulation energy and the second neuromodulation energy, wherein the first neuromodulation field is configured to stimulate a first nerve tract without stimulating a second nerve tract, and the second neuromodulation field is configured to stimulate the second nerve tract without stimulating the first nerve tract; or the first neuromodulation field is at a first spinal cord level without extending to a second spinal cord level, and the second neuromodulation field is at the second spinal cord level without extending to the first spinal cord level.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver neurostimulation such as SCS.

FIG. 2 illustrates, by way of example and not limitation, a computing device for programming or controlling the operation of an electrical stimulation system.

FIG. 3 illustrates, by way of example and not limitation, a more generalized example of a medical system that includes a medical device and a processing system.

FIG. 4 illustrates, by way of example, an example of an electrical therapy-delivery system.

FIG. 5 illustrates, by way of example and not limitation, the electrical therapy-delivery system of FIG. 4 implemented using an SCS device to provide epilepsy therapy(ies).

FIG. 6 illustrates, by way of example and not limitation, neural targets for an epilepsy therapy.

FIG. 7 illustrates a trigeminocervical nucleus where trigeminal nerve sensory afferents (V1, V2 and V3) and cervical nerve root (C1, C2, and C3) afferents converge and send signals to the thalamus via the trigeminothalamatic tract.

FIG. 8 illustrates the regions of the face that are innervated by the branches of the trigeminal nerve.

FIGS. 9A-9B illustrate, by way of example, and not limitation, a representation of some anatomical features of a brain and spinal cord of a human.

FIGS. 10A-10B illustrate a representation of a thalamus.

FIG. 11 illustrates, by way of example and not limitation, a portion of a spinal cord and various nerve tracts found within the spinal cord white matter.

FIG. 12 illustrates a view of the spinal cord without the bony anatomy.

FIGS. 13A-13C illustrate, by way of example and not limitation, various electrode arrangements that may be used to stimulate various spinal tracts within the spinal cord.

FIGS. 14A-14C illustrate examples of directional leads with segmented electrodes that are circumferentially and axially disposed about the leads.

FIG. 15A illustrates a portion of the spinal cord and illustrates a neural stimulation lead fed through the dorsal epidural space (behind the illustrated cord) and least partially around the spinal cord to operationally set electrodes in place to stimulate activity in at least some nerve pathways on the lateral side of the spinal cord; and FIG. 15B illustrates a multi-lead embodiment to stimulate neural targets on contralateral sides of the spinal cord.

FIG. 16 illustrates a nerve tract with a plurality of pathways (not to scale) and neuromodulation field(s) focused to modulate some of the plurality of pathways in the nerve tract.

FIG. 17 illustrates, by way of example and not limitation, a neuromodulation device with a plurality of different programs for delivering neuromodulation.

FIG. 18 illustrates, by way of example and not limitation, a therapy device with a sensing function.

FIG. 19 illustrates, by way of example and not limitation, an external system with sensing function.

FIG. 20 illustrates, by way of example and not limitation, a method for delivering an epileptic therapy in response to a detected event.

FIG. 21 illustrates, by way of example and not limitation, a system for treating epilepsy using at least one event detector.

FIG. 22 illustrates, by way of example and not limitation, a system configured for defining event-therapy relationship(s) that associates therapies to detected events.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter provides a system for treating epilepsy that less invasive than implanted DBS and VNS systems, and that is configurable to stimulate any one or more of multiple neural pathways to treat epilepsy. The present subject matter proposes using an SCS system to treat epilepsy. SCS is less invasive than DBS and VNS and the surgical implantation procedure is less complicated for SCS surgery than DBS and VNS. SCS may be delivered in the cervical or thoracic regions to stimulate nerve pathways (e.g., axons and/or nuclei) in or near the spinal cord that transmit impulses (e.g., signals) to the cerebellum or the thalamus. A 1982 publication indicated that two patients became seizure free with SCS, including a patient who suffered 10-15 absence attacks per day and 3-4 grand mal seizures per week and frequent drop attacks became totally free of all seizures. 1982 J. Waltz, Computerized Percutaneous Multi-Level Spinal Cord Stimulation in Motor Disorders, Appl. Neurophysiol. Preclinical studies indicate cervical placement is effective (Ozcelik et al. 2007, Harreby et al. 2011, Jia et al. 2015, Pais-Vieira et al. 2016). Another preclinical study suggests thoracic may be even more effective (Ye et al. 2013). Frequencies within a 130 and 180 Hz was shown to be more effective than a range of between 30 and 80 Hz (Jiao et al. 2015). A 500 Hz burst has been shown to be effective (Pais-Vieira et al. 2016). In rats, the DCN (Dorsal Column Nuclei) extend their axons to the VPL (ventroposterolateral nucleus of the thalamus) which is part of the somatosensory thalamus and also to the posterior thalamic group. Also, a separate portion of the axons splits off the medial lemniscus at the obex to terminate in the pontine nuclei, parabrachial nuclei, dorsal reticular nuclei, and the inferior colliculus, as well as the mesencephalic reticular nuclei. There are also projections to the inferior olive, and cerebellum. 2004 David Tracey, “The Rat Nervous System” (Third edition).

The intralaminar nuclei are a group of nuclei located within the thalamus of the brain. These nuclei have complex and multifaceted functions. While their exact roles are not completely understood, they are known to be involved in various processes related to sensory-motor integration, arousal, attention, and cognitive functions. Their role in sensory-motor integration implies involvement in integrating sensory information from various sources and connecting it with motor responses allowing for the coordination of movements and responses to sensory stimuli. Additionally, the posterior intralaminar nuclei appear to provide a significant role for speaking and motivation. The centromedian (CM) nucleus is part of the posterior (caudal) intralaminar nuclei that receives projections from the DCN. DBS treatments for epilepsy often target the CM. Interesting the intralaminary nuclei are contained within the Y-shaped vertical sheet of white matter (axons) within the thalamus, called the internal medullary lamina. Several fibers in the internal medullary lamina traverse through the lamina to interconnect the various thalamic nuclei with each other. The Y-shape sheet of axons pretty much travels in proximity to all the thalamus nuclei. If the DCN projects axons into the intralaminary nuclei, then it is believed to be likely that the DCN can send information to all the thalamic nuclei

Neuromodulation energy may be delivered as a prophylactical therapy to avoid epileptic conditions. The prophylactic neuromodulation may be delivered continuously or may be delivered intermittently according to a schedule. Some embodiments may initiate neuromodulation energy when seizure activity is detected. Closed-loop stimulation using signals like brain activity, cardiac activity, accelerometry, or video analysis of motion signatures may be used to control timing of the neuromodulation energy as well as various temporal and spatial parameters of the delivered energy. The stimulation may be delivered to select neural pathways to signal specific regions of the brain. Thus, for example, the region of the brain where a focal seizure occurs may be targeted. Some seizures may spread from one part of the brain to others. For example, a secondary generalized seizure may begin in one part of the brain and spread to affect both sides of the brain. The stimulation may be delivered in a manner timed to prevent the spread. The seizure may cause symptoms in other parts of the patient's body. The targeted neural pathway may be determined based on where the patient experiences a symptom of the epileptic seizures. It is believed that undesired side effects may be avoided by focusing the stimulation to neural pathways that only signal the part(s) of the brain that experience seizure.

Different therapies, alone or in combination with each other, may be implemented in response to different conditions. These different therapies may be delivered to different neural targets and may provide different mechanisms of action to treat a condition. Therapy delivery may be coordinated to address different detected events. Temporal parameters (e.g., frequency, pulse width, stimulation burst duration for a train of pulses, stimulation on/off timing and the like) and/or spatial parameters (e.g., stimulation amplitude, activated electrodes, polarity of active electrodes, and distribution of energy (fractionalization) across the active electrodes, and the like) for therapies may be adjusted using inputs (e.g., sensed parameters and/or user inputs) into the system.

For example, two or more therapies may be applied to a patient who has epilepsy. The therapies may be selected and timed to ameliorate the patient condition (e.g., interrupt the progression of patient states that may develop into a full seizure). For example, a first therapy may be provided when a first state of the epileptic patient is detected, and a second therapy may be provided when a second state of the epileptic patient is detected. In another example, a therapy may be delivered upon the detection of a seizure or known precursor to a seizure, and another therapy may be delivered upon the termination of the seizure. In another example, a first therapy may be delivered prophylactically to reduce the number or severity of seizures, a second therapy may be delivered to reduce the duration or intensity of a seizure that is currently occurring, and the third therapy may be delivered after the seizure has terminated before returning the prophylactic therapy again.

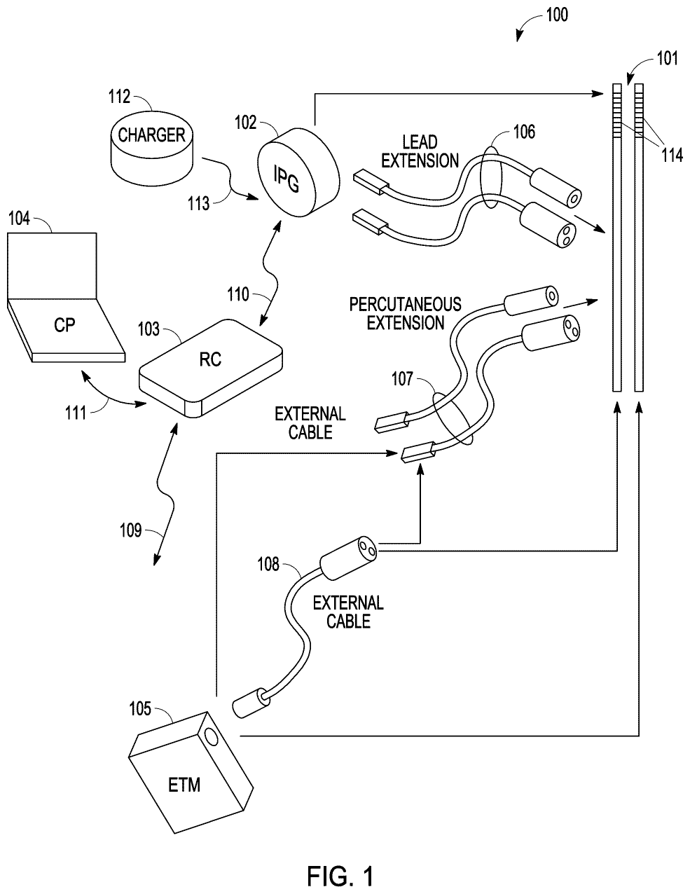

FIG. 1 illustrates, by way of example and not limitation, an electrical stimulation system 100, which may be used to deliver neurostimulation such as SCS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 114. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 102 includes pulse generation circuitry that delivers electrical modulation energy in the form of a pulsed electrical waveform (i.e., a temporal series of electrical pulses) to the electrodes in accordance with a set of modulation parameters.

The IPG 102 may include an antenna allowing it to communicate bi-directionally with a number of external devices. The antenna may be a conductive coil within the case, although the coil of the antenna may also appear in the header. When the antenna may be configured as a coil, communication with external devices may occur using near-field magnetic induction. The IPG 102 may also include a Radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a map that shows which areas of the body are affected by epilepsy, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed and its power source has been charged by the external charger or otherwise replenished, the IPG 102 may function as programmed without the RC 103 or CP 104 being present.

The waveform generator may generate non-pulsatile waveforms or waveforms comprised of non-rectilinear pulses. However, the IPG 102 typically provides pulses each of which may include one phase or multiple phases. For example, a monopolar stimulation current can be delivered between a lead-based electrode (e.g., one of the electrodes) and a case electrode. A bipolar stimulation current can be delivered between two lead-based electrodes (e.g., two of the electrodes). Stimulation parameters typically include current amplitude (or voltage amplitude), frequency, pulse width of the pulses or of its individual phases, electrodes selected to provide the stimulation (i.e., “active electrodes”) and polarity of such selected electrodes (i.e., whether they act as anodes that source current to the tissue or cathodes that sink current from the tissue). Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry in the IPG can execute to provide therapeutic stimulation to a patient.

Various embodiments may use multiple independent current control (MICC) technology to achieve better selectivity and directionality to recruit the appropriate neural elements. MICC technology may provide an independent current source (e.g., including a positive or anodic current source and a negative or cathodic current source) for each electrode or each active electrode being used to deliver the neuromodulation. The independent current sources may be controlled such that the total amount of anodic current may be distributed across one or more electrodes and the total amount of cathodic current may be distributed across one or more electrodes. MICC technology is capable of focusing a neuromodulation field on a targeted neural pathway as it is capable of being used to select electrodes to be active, select the polarity of individual ones of active electrodes, and select the fractionalized distribution of energy using the active electrodes.

MICC technology allows multiple stimulation targets in or near the spine that transmit impulses to the cerebellum or thalamus are capable of being modulated. In some examples, a measurement device may be used to detect when target neurons are stimulated or to confirm that non-targeted neurons are not stimulated to avoid undesired side effects of the stimulation. The specific measurement may depend on the specific neurons of interest. Alternatively, the patient or clinician can observe stimulation effects and provide feedback. Neuromodulation systems may be configured to independently modulate more than one neural target. According to various embodiments, the neuromodulation system may be configured to coordinate the modulation to these different targets, such as may appropriate in response to different detected events.

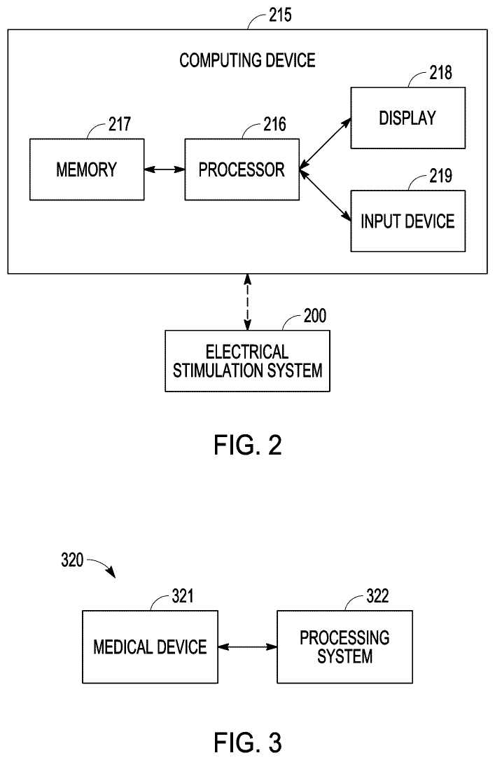

FIG. 2 illustrates, by way of example and not limitation, a computing device 215 for programming or controlling the operation of an electrical stimulation system 200. The computing device 215 may include a processor 216, a memory 217, a display 218, and an input device 219. Optionally, the computing device 215 may be separate from and communicatively coupled to the electrical stimulation system 200. Alternatively, the computing device 215 may be integrated with the electrical stimulation system 200, such as part of the IPG 102, RC 103, CP 104, or ETM 105 illustrated in FIG. 1.

The computing device 215, also referred to as a programming device, can be a computer, tablet, mobile device, or any other suitable device for processing information. The computing device 215 can be local to the user or can include components that are non-local to the computer including one or both of the processor 216 or memory 217 (or portions thereof). For example, the user may operate a terminal that is connected to a non-local processor or memory. The functions associated with the computing device 215 may be distributed among two or more devices, such that there may be two or more memory devices performing memory functions, two or more processors performing processing functions, two or more displays performing display functions, and/or two or more input devices performing input functions. In some examples, the computing device 215 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 215 may be used for gathering patient information, such as general activity level or present queries or tests to the patient to identify or score stimulation effects or side effects, physical state or the like. In some examples, the computing device 215 may detect, or otherwise receive as input, patient clinical responses to neurostimulation, and determine or update stimulation parameters using a closed-loop algorithm based on the patient clinical responses. Examples of the patient clinical responses may include physiological signals (such as but not limited heart rate/rhythm, brain activity, and muscle activity), audio signals (e.g., sound) or visual signals (e.g., camera). The computing device 215 may communicate with the CP 104, RC 103, ETM 105, or IPG 102 and direct the changes to the stimulation parameters to one or more of those devices. In some examples, the computing device 15 can be a wearable device used by the patient only during programming sessions. Alternatively, the computing device 215 can be worn all the time and continually or periodically adjust the stimulation parameters. In an example, a closed-loop algorithm for determining or updating stimulation parameters can be implemented in a mobile device, a wristband or watch. These devices can also record and send information to the clinician.

The processor 216 may include one or more processors that may be local to the user or non-local to the user or other components of the computing device 215. A stimulation setting (e.g., modulation parameter set) may referred to as a stimulation configuration. The stimulation configuration has a spatial component that defines where to stimulate and has a temporal component that defines a waveform (e.g., regular or irregular pulse pattern). The spatial component of the stimulation configuration may include an electrode configuration that includes information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, a percentage of modulation energy assigned to each electrode (fractionalized electrode configurations), and the like. The temporal component of the stimulation configuration may include electrical pulse parameters, which define the pulse amplitude (e.g., measured in milliamps or volts depending on whether the IPG is configured with current or voltage sources), pulse duration (e.g., measured in microseconds), pulse rate (measured in pulses per second), and burst rate (measured as the modulation on duration X and modulation off duration Y). The temporal component may be defined with other parameters as appropriate for the energy waveform that is being delivered to stimulate the neural target. The stimulation configuration may include a plurality of pulses in a pulse pattern. The pulse pattern may not be a regular pattern by including different pulse amplitudes, different pulse widths, different pulse-to-pulse spacing, different pulse shapes or different bursts of pulses.

The processor 216 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 216 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 217 can store instructions executable by the processor 216 to perform various functions. The memory 217 may store the search space, the stimulation settings including the “tested” stimulation settings and the “predicted” or “estimated” stimulation settings, clinical effects (e.g., therapeutic effects and/or side effects) and clinical responses for the settings. The memory 217 may be a computer-readable storage media that includes, for example, nonvolatile, non-transitory, removable, and non-removable media implemented in any method or technology for storage of information.

The display 218 may be any suitable display or presentation device, such as a monitor, screen, display, or the like, and can include a printer. The display 218 may be a part of a user interface configured to display information about stimulation settings (e.g., electrode configurations and stimulation parameter values and value ranges) and user control elements for programming a stimulation setting into a neuromodulator. The input device 219 may be, for example, a keyboard, mouse, touch screen, track ball, joystick, voice recognition system, or any combination thereof, or the like. Another input device 219 may be a camera from which the clinician can observe the patient. Yet another input device 219 may include a microphone where the patient or clinician can provide responses or queries or can otherwise monitor sounds made by the patient or made in an environment of the patient.

The electrical stimulation system 200 may include, for example, any of the components illustrated in FIG. 1. The electrical stimulation system 200 may communicate with the computing device 215 through a wired or wireless connection or, alternatively or additionally, a user can provide information between the electrical stimulation system 200 and the computing device 215 using a computer-readable medium or by some other mechanism.

FIG. 3 illustrates, by way of example and not limitation, a more generalized example of a medical system 320 that includes a medical device 321 and a processing system 322. For example, the electrical stimulation system 200 of FIG. 2 may be a more specific example of the medical system 320 of FIG. 3, and computing device 215 of FIG. 2 may be a more specific example of the processing system 322 of FIG. 3. The medical device may be configured to provide sensing functions and/or therapy functions. For example, the medical device may include a device configured to use a parameter set to deliver an electrical stimulation therapy. The medical device may be an implantable medical device such as an implantable neurostimulator. The medical device may include more than one medical device. The processing system may be within a single device or may be a distributed system across two or more devices including local and/or remote systems. According to various embodiments, the medical system may include at least one medical device configured to treat a condition (e.g., epileptic seizures) by delivering a therapy to a patient.

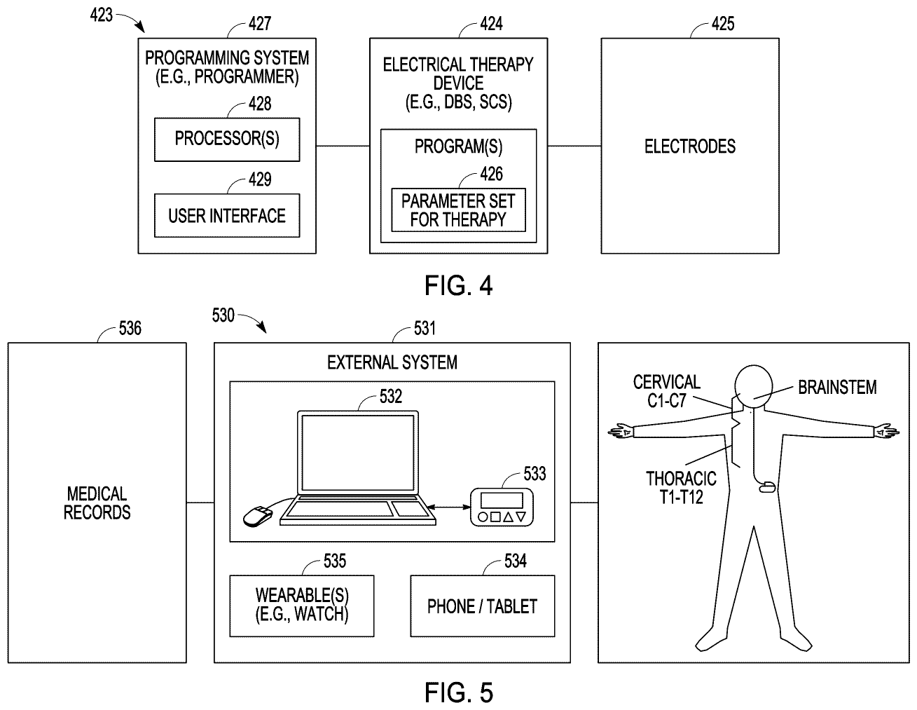

FIG. 4 illustrates, by way of example, an example of an electrical therapy-delivery system. The illustrated system 423 may be a more specific example of the system illustrated in FIG. 3 or form a portion of the system illustrated in FIG. 3. The illustrated system 423 includes an electrical therapy device 424 configured to deliver an electrical therapy to electrodes 425 to treat a condition (e.g., epileptic seizure) in accordance with a programmed parameter set 426 for the therapy. The system 423 may include a programming system 427, which may function as at least a portion of a processing system. The programming system may include one or more processors 428 and a user interface 429. The programming system 427 may be used to program and/or evaluate the parameter set(s) used to deliver the therapy.

A therapy may be delivered according to a parameter set that defines a stimulation configuration. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 5 illustrates, by way of example and not limitation, the electrical therapy-delivery system of FIG. 4 implemented using an SCS device to provide epilepsy therapy(ies). The therapy(ies) may direct neuromodulation energy fields to at least one neural target, which may include neural pathway target(s) in the cervical region (e.g., C1-C7), neural pathway target(s) in the thoracic region (e.g., T1-T12), or neural pathway target(s) in the brainstem such as the dorsal column nuclei at the junction between the brain stem and the spinal cord. The dorsal column nuclei and the axons projecting to the dorsal column nuclei are somatotopically organized and project to at least one of a cerebellum, a thalamus or another brainstem area. It is noted the dorsal column nuclei are part of the medulla, which is part of the brainstem. The cerebellum, the thalamus or the brainstem include somatotopically organized nuclei and project axons to different areas of a cortex and/or limbic system. The limbic system includes: amygdala, hippocampus, thalamus, hypothalamus, basal ganglia, and cingulate gyrus. The cingulate gyrus in part of the cingulate cortex. Pathways that tap into the thalamus or cortex may engage the other parts of the limbic system. The limbic system may be involved in several type of focal seizures and in generalized seizures, and the thalamus is a window to it. The hippocampus is part of the same network.

Different therapies (e.g., different stimulation parameters such as but not limited to different waveform patterns) may be delivered to the same neural target. Two or more distinct neural targets may be modulated. Therapies may be coordinated or may be independently controlled. The therapies may respond to the detection of one more events. The illustrated system 530 includes an external system 531 that may include at least one programming device. The illustrated external system 531 may include a clinician programmer 532, similar to CP 104 in FIG. 1, configured for use by a clinician to communicate with and program the neuromodulator, and a remote control device 533, similar to RC 103 in FIG. 1, configured for use by the patient to communicate with and program the neuromodulator. For example, the remote control device 533 may allow the patient to turn a therapy on and off, change or select programs, and/or may allow the patient to adjust patient-programmable parameter(s) of the plurality of modulation parameters. The monitor and/or therapy device may be implemented using an implantable medical device and/or an external device such as a wearable device. The external system 531 may include a network of computers, including computer(s) remotely located from the IMD that are capable of communicating via one or more communication networks with the programmer 532 and/or the remote control device 533. The remotely located computer(s) and the IMD may be configured to communicate with each other via another external device such as the programmer 532 or the remote control device 533. The remote control device 533 and/or the programmer 532 may allow a user (e.g., patient, caregiver and/or clinician or rep) to answer questions as part of a data collection process. The external system 531 may include personal devices such as a phone or tablet 534, wearables such as a watch 535, sensors or therapy-applying devices. The watch may include sensor(s), such as sensor(s) for detecting activity, motion and/or posture. Other wearable sensor(s) may be configured for use to detect activity, motion and/or posture of the patient. A camera, such as a camera in a phone or tablet, may be used to capture images of the patient. A microphone may be used to detect sound associated with various events (e.g., a type of epileptic seizure) for the patient. These sensors (including a camera and microphone) may be used to detect seizures or other events associated with the epilepsy of the patient. These detected event(s) may be used by the system to trigger a therapy or trigger a change in the therapy. These sensors and camera may also be used to determine the effectiveness of a therapy, such as may be useful in a machine learning algorithm used to develop effective stimulation patterns for an epilepsy therapy. The system 530 may include medical record(s) 536 for the patient and broader patient population(s). The medical record(s) may be stored and accessed using one or more servers (e.g., local or remote servers such as cloud-based servers). Medical information from these medical records may be inputted into the machine learning algorithm to develop the effective stimulation parameters, which may include both an effective stimulation location and an effective temporal stimulation pattern delivered at the effective stimulation location.

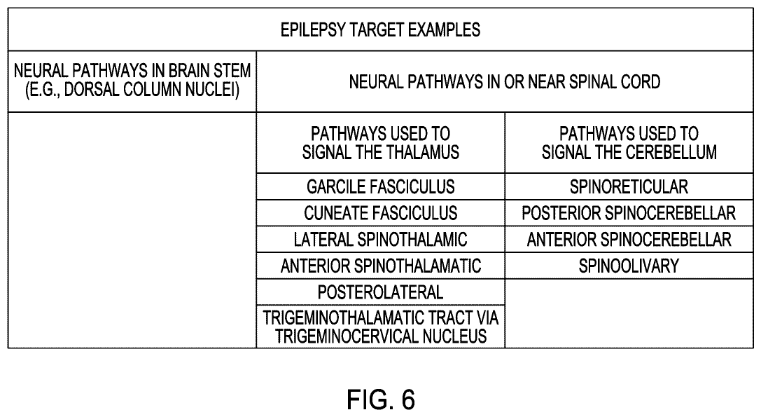

FIG. 6 illustrates, by way of example and not limitation, neural targets for an epilepsy therapy. Generally, the targets are accessible using a lead fed into an epidural space toward the superior end of the spine with electrodes that may span at least portions of the cervical and thoracic layers of the spine. The targets may include neural target(s) in the brainstem such as but not limited to the dorsal column nuclei, and/or neural target(s) in or near the spinal cord. For example, some tracts within the spinal cord that may be targeted and used to signal the cerebellum and some tracts within the spinal cord that may be targeted used to signal the thalamus. Targeted spinal cord tracts that may be used to signal the thalamus may include one or more of the gracile fasciculus, the cuneate fasciculus, the lateral spinothalamic, the anterior spinothalamatic, the posterolateral and/or the trigeminothalamatic tract via the trigeminocervical nucleus. Targeted spinal cord tracts that may be used to signal the cerebellum may include one or more of the spinoreticular tract, the posterior spinocerebellar tract, the anterior spinocerebellar tract and the spinoolivary tract.

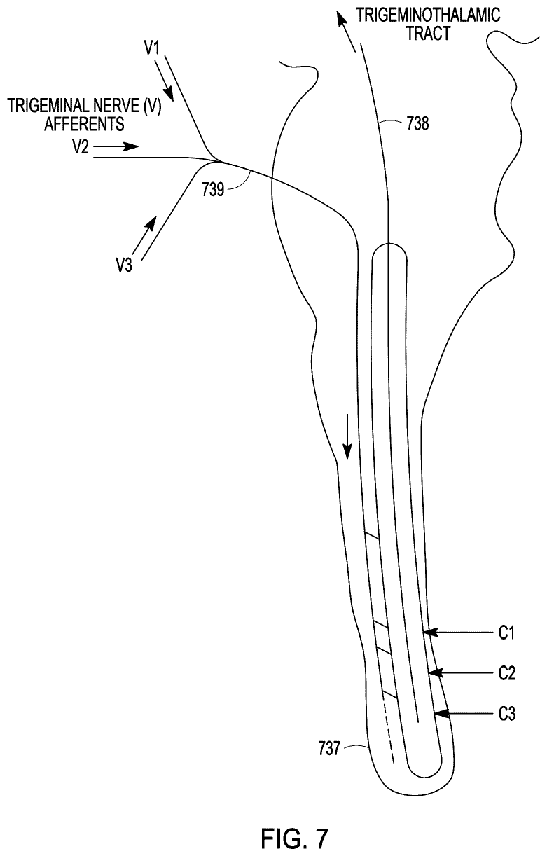

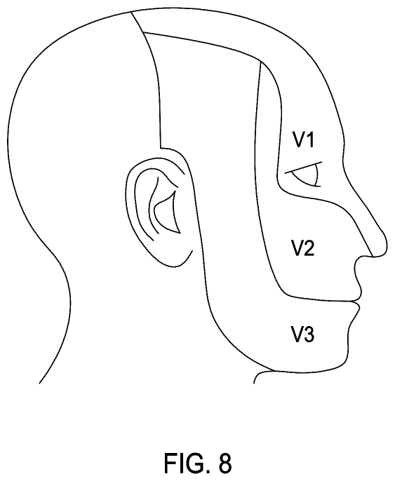

FIG. 7 illustrates a trigeminocervical nucleus 737 where trigeminal nerve sensory afferents (V1, V2 and V3) and cervical nerve root (C1, C2, and C3) afferents converge and send signals to the thalamus via the trigeminothalamatic tract 738. The trigeminal nerve 739 is the fifth cranial nerve, and branches of the trigeminal nerve 739 include an ophthalmic nerve (V1), the maxillary nerve (V2) and the mandibular nerve (V3). FIG. 8 illustrates the regions of the face that are innervated by these three nerves. The ophthalmic nerve (V1) is a sensory nerve conveying senses from the upper part of the face (e.g., eyelids and supraorbital region of the face), the maxillary nerve (V2) is a sensory nerve conveying senses from the mid part of the face, and the mandibular nerve (V3) includes both sensory branches for the lower part of the face and motor branches. The sensory nerves are afferent pathways sending signals toward the brain.

The trigeminal nerve 739 descends toward the brainstem and splits into ascending and descending fibers in the pons. The descending fibers join the trigeminocervical nucleus 737. Both pain from the neck region received via the C1-C3 afferents and pain from the face received via the trigeminal nerve sensory afferents V1-V3 are sent to the brain (e.g., thalamus) through the trigeminothalamic tract 738. The trigeminothalamic tract include a ventral trigeminothalamic tract that projects to the contralateral ventral posteromedial nucleus of the thalamus (VPM), and a dorsal trigeminothalamic tract projects to the ipsilateral VPM. The VPM can send pain signals to the intralaminar thalamic nuclei, which conveys the signal to the cortex.

The trigeminocervical nucleus 737 extends through the brainstem and into the cervical spinal cord. Various embodiments may stimulate afferent pathways that feed into the trigeminocervical complex to treat epilepsy. Electrical stimulation energy may be epidurally applied by the stimulation lead(s) to one or more of the C1-C3 segments of the spinal cord to treat epilepsy. It may be desirable to avoid inadvertently creating side-effects, e.g., in the form of uncomfortable muscle contractions and pain resulting from the inadvertent stimulation of the DR and/or VR nerve fibers. Thus, the electrodes may be arranged to avoid capturing the VR nerve fibers and/or DR nerve fibers. Alternatively, or additionally, inadvertent stimulation of the DR and/or VR nerve fibers may be avoided by increasing the activation threshold of the VR nerve fibers and/or the DR nerve fibers relative to the spinal cord. Some embodiments may include a lead subcutaneously tunneled to the trigeminal nerve in or near the pons or to one or more of the trigeminal nerve branches in the V1, V2 or V3 regions.

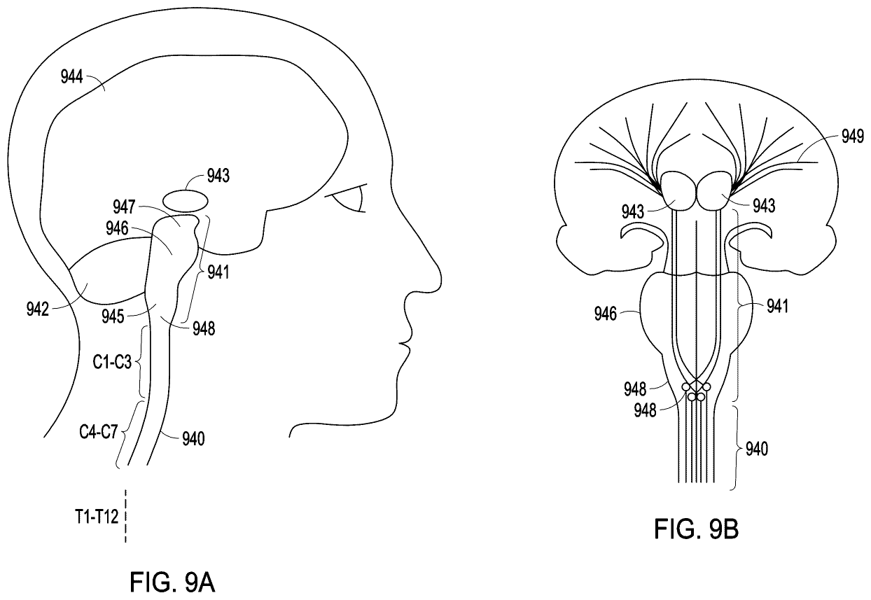

FIGS. 9A-9B illustrate, by way of example, and not limitation, a representation of some anatomical features of a brain and spinal cord of a human. The upper portion of the spinal cord 940 includes a cervical segment (C1-C7), which includes segments C1-C3 which include afferents to the trigeminocervical nucleus as well as segments C4-C7. The spinal cord 940 includes a thoracic segment (T1-T12) below the cervical segments, and also includes lumbar segments (not illustrated). Various embodiments may stimulate spinal tracts that transmit impulses to either the thalamus or the cerebellum along the segments C1 through T12. FIGS. 9A-9B also illustrate a brainstem 941, cerebellum 942, a thalamus 943 and a cerebrum 944. The brainstem 941 includes a medulla oblongata 945, a pons 946, and a midbrain 947. FIG. 9B illustrates dorsal column nuclei (DCN) 948, which are cuneate and gracile nuclei located at the junction between the medulla oblongata 945 and the cervical region of the spinal cord. The cuneate and gracile tracts (also referred to as the dorsal column) carry information about tactile sensations and proprioception (ability to sense movement, action, location). These tracts synapse at the DCN. The information (impulses) contained in the cuneate and gracile tracts is passed to the medial lemniscus and then to the thalamus. The DCN project to the thalamus. The axons in the dorsal column synapse at the DCN and are used to provide signals to the thalamus. Axons from the DCN cross over the midline at the sensor decussation and enter the contralateral medial lemniscus. Some DCN axons project to the thalamus 943 (specifically the ventral posterior lateral nucleus and ventral posterior medial nucleus), which are part of the somatosensory pathway, and various nuclei in the thalamus 943 project axons 949 to different areas of the cerebral cortex.

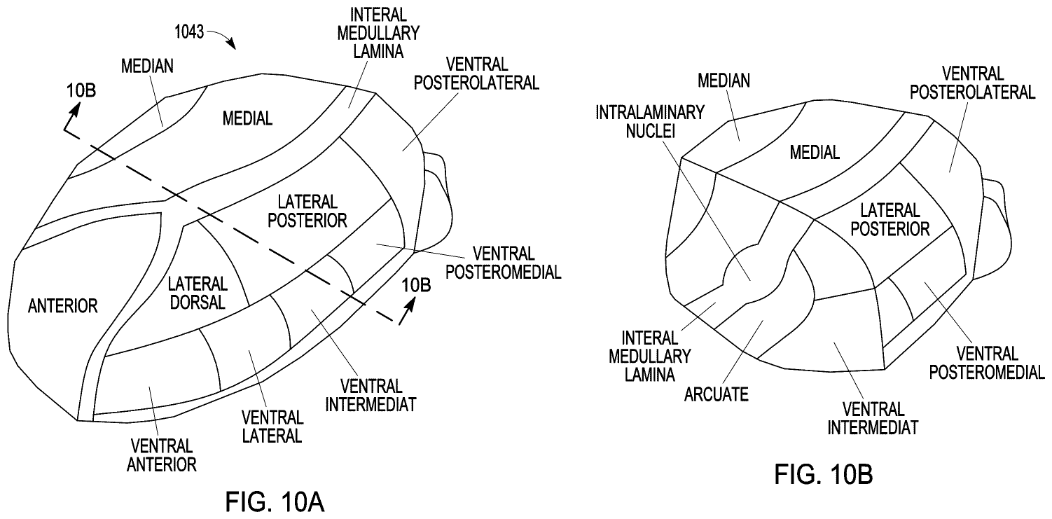

FIGS. 10A-10B illustrate a representation of a thalamus. More particularly, FIG. 10A is a perspective view of the thalamus and FIG. 10B is a view taken along line 10B-10B in FIG. 10A. The thalamus 1043 is located above the brainstem and in the middle of the brain, and it functions to relay incoming motor and sensory information toward the cerebral cortex. The thalamus prioritizes received information and contributes to sleep, consciousness, thinking and memory. The various nuclei in the thalamus have different functions and project to different areas of the cerebral cortex. With reference to FIGS. 6, 7, 10A-10B, and 11, the following provides some examples of the nerve pathways that transmit impulses from the spinal cord to various regions of the brain.

The gracile fasciculus projects to the ventroposterolateral nucleus of the thalamus, which then projects to the somesthetic area of the postcentral gyrus of the cerebral cortex. The cuneate fasciculus projects to the ventroposterolateral nucleus of the thalamus, which then projects to the lateral aspect of the sensorimotor cortex, and a few fibers from the cuneate fasciculus projects to the cerebellum as the cuneocerebellar tract and conveys muscle joint sense information to the cerebellum. Many of the nerve fibers in the spinothalamatic tract project to the ventral posterolateral nucleus, which then project to the postcentral gyrus of the cerebral cortex. The spinocerebellar tract projects to the cerebellum. The anterior spinocerebellar tract also projects to the cerebellum. The trigeminothalamatic tract projects to the ventral posteromedial nucleus as discussed below.

The anterior nucleus is associated with memory, emotions, and behavior, and sends signals to the hypothalamus and the cingulate gyrus. The dorsal nucleus is associated with emotional behavior and memory, attention, organization, planning and higher cognitive thinking, and sends signals to the prefrontal cortex and limbic system. The ventral posterolateral nucleus relays sensory information and signals the somatosensory cortex. The ventral posteromedial nucleus relays sensory information from the face and sends signals to the somatosensory cortex. The ventral anterior nucleus relays motor information about movement/tremor and sends signals to the substantia nigra, premotor cortex, reticular formulation and corpus striatum. The ventrolateral nucleus relays motor information and sends signals to the substantia nigra, premotor cortex, reticular formulation and corpus striatum. The lateral posterior nucleus sends signals to the visual cortex. The pulvinar nucleus is associated with visual information and sends signals to the visual cortex. The medial geniculate nucleus is associated with auditory information and sends signals to the primary auditory cortex. The lateral geniculate nucleus associates visual information and sends signals to the visual cortex. The reticular nucleus (outer covering of thalamus influences the activity of other nuclei within the thalamus. (https://my.clevelandclinic.org/health/body/22652-thalamus).

The internal medullary lamina of the thalamus is white matter (axons) that interconnect thalamic nuclei with each other. The Y-shaped internal medullary lamina splits the gray matter of the thalamus into various parts (medial, lateral and anterior group)s. The DCN do not directly send axons to the intralaminar nuclei of the thalamus. The intralaminar nuclei, on the other hand, are a distinct group of nuclei within the thalamus that have roles in various functions such as arousal, attention, and sensory-motor integration. They receive inputs from multiple sources, including the basal ganglia and brainstem reticular formation, rather than directly from the dorsal column nuclei. The posterior group of intralaminar nuclei receive afferent fibers from the internal segment of globus pallidus, pars reticulata of substatia nigra, deep cerebellar nuclei, pedunculopontine nucleus of midbrain and the spinothalamic tract. Kenhub (https://www.kenhub.com/en/library/anatomy/thalamic-nuclei).

It is believed to be likely that the ventral posteromedial and ventral posteromedial that receive pathways from the DCN are likely connected to the intralaminar nuclei because thalamic nuclei are interconnected with each other through the internal medullary lamina. And if the intralaminar nuclei receives pathways from the nociceptive spinal cord via the spinothalamic tract, then this information is integrated with other thalamic nuclei through the internal medular lamina.

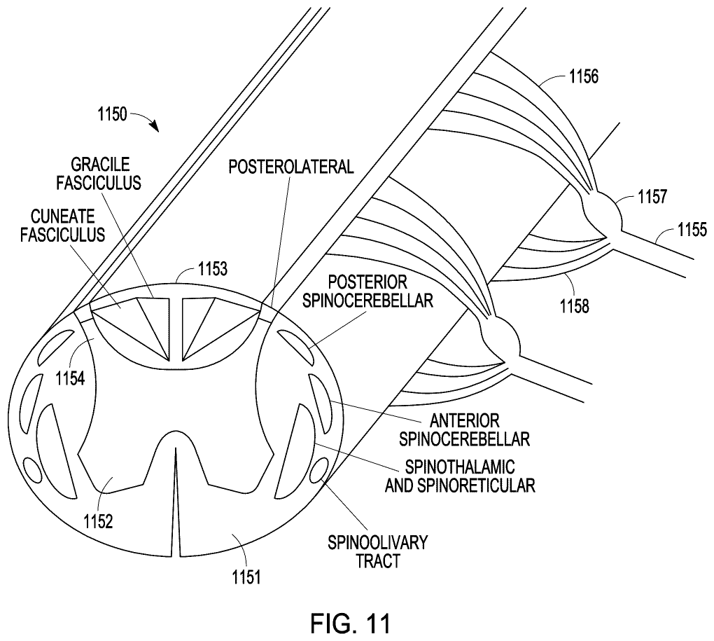

FIG. 11 illustrates, by way of example and not limitation, a portion of a spinal cord and various nerve tracts found within the spinal cord white matter. The spinal cord 1150 includes white matter 1151 and gray matter 1152. The gray matter 1152 includes cell bodies, synapse, dendrites, and axon terminals. Thus, synapses are located in the gray matter. White matter 1151 includes myelinated axons that connect gray matter areas. A typical transverse section of the spinal cord includes a central “butterfly” shaped central area of gray matter 1152 substantially surrounded by an ellipse-shaped outer area of white matter 1151. The white matter of the dorsal column (DC) 1153 includes mostly large, myelinated axons that form afferent fibers that run in an axial direction. The dorsal portions of the “butterfly” shaped central area of gray matter are referred to as dorsal horns (DH) 1154. Examples of spinal nerves 1155 are also illustrated, including a dorsal root (DR) 1156, dorsal root ganglion 1157 and ventral root 1158. The dorsal root 1156 mostly carries sensory signals into the spinal cord, and the ventral root 1158 functions as an efferent motor root. The dorsal and ventral roots join to form mixed spinal nerves 1155. Sensory nerve fibers form bundles within the spinal cord according to their function. These bundles are found within the white matter 1151 of the spinal cord and may be referred to nerve tracts or fasciculi. Ascending tracts within the white matter 1151 transmit information from the periphery to the brain and descending tracts within the white matter 1151 transmit information from the brain to the periphery. Various ascending tracts run along the dorsal, lateral, and ventral columns of the white matter and mainly transmit somatosensory information. However, the precise position of some tracts is still not known with certainty. The somatotopic organization of the fibers in the spinal cord are maintained throughout, as the fibers from the sacral region are more lateral while those from the upper limb and cervical region are more medial.

The fasciculus gracilis begins at the caudal end of the spinal cord. The fibers enter the spinal cord at the sacral, lumbar and lower six thoracic nerves. The first order neurons entering through dorsal root of spinal nerves brings tactile, vibratory and proprioceptive information mainly from the lower body and terminate on to the second order neurons in the ipsilateral nucleus gracilis. Axons of second order neuron cross in the midline to form medial lemniscus, pass through the medulla, pons and midbrain and terminate in third order neurons at the ventral posterolateral nucleus of thalamus. The axons of these third order neurons project to the somesthetic area of the postcentral gyrus of the cerebral cortex. The fasciculus cuneatus begins at the mid-thoracic level of the spinal cord. The fibers enter the spinal cord at the upper thoracic and cervical dorsal roots of the spinal cord, and carry tactile, vibratory and proprioceptive information from the upper body. The fibers terminate on the second order neurons of the nucleus cuneatus. These fibers also cross at the midline and terminate as third order neurons at the ventral posterolateral nucleus of thalamus. The axons of these third order neurons project to the cerebral cortex and terminate at the lateral aspect of sensorimotor cortex. Some second order fibers from the nucleus cuneatus terminate in the accessory cuneate nucleus in the medulla oblongata at the level of the sensory decussation, and these axons enter the cerebellum through the inferior cerebellar peduncle as the cuneocerebellar tract, which carries muscle joint sense information to the cerebellum. The spinothalamic tracts carry pain, temperature, non-discriminative touch and pressure information to the thalamus. The cell bodies of origin are located in spinal gray matter. The spinothalamic tract ascends the medulla oblongata along with the spinotectal and anterior spinothalamic tracts (referred to as the spinal lemniscus). Many lateral spinothalamic tract fibers synapse onto the third order neurons in the ventral posterolateral nucleus of the thalamus, and the axons of these third order neurons project to the postcentral gyrus of the cerebral cortex. The anterior spinothalamic tract carries light touch and pressure sensation information and are also somatotopically arranged such that the sacral and lumbar fibers are lateral while those from the thoracic and cervical regions are medial. The first order neurons of the anterior spinothalamic tract are found in the dorsal root ganglia of the spinal nerves, the second order neurons are found between laminae IV and VI in the posterior horn, and the axons of the second order neurons form the anterior spinothalamic tract which project to the ventral posterolateral nucleus of the thalamus, which sends impulses to the postcentral gyrus of the brain's cerebral cortex. The posterior spinocerebellar tract begins in the L2-3 region, and carries proprioception, cutaneous touch and pressure information from the lower limb and trunk. The first order neurons terminate by synapsing with second order neurons in the posterior grey column of the spinal cord located at cord segments C8-T1 through L2-3. The fibers reach the medulla and then enters the cerebellum. The anterior spinocerebellar tract carries unconscious proprioception from the lower limb. After passing through the medulla and pons, the fibers terminate in the vermal and paravermal regions of the cerebellum.

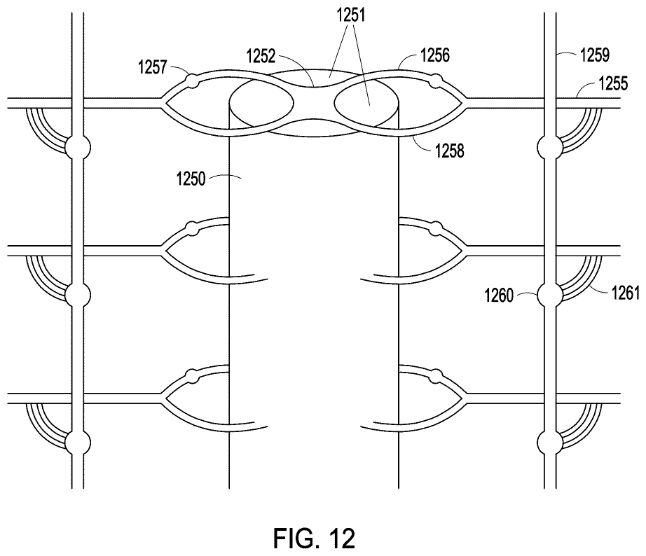

FIG. 12 illustrates a view of the spinal cord 1250 without the bony anatomy. The figure illustrates white matter 1251 and gray matter 1252. Examples of spinal nerves 1255 are also illustrated, including a dorsal root 1256, dorsal root ganglion 1257 and ventral root 1258. FIG. 12 also illustrates sympathetic chains 1259 with ganglia 1260 and the white and gray rami 1261 connecting the sympathetic chains 1259 to spinal nerves 1255. Various embodiments configure the system to stimulate the sympathetic chains. For example, electrodes can be laterally placed and oriented to focus stimulation toward the sympathetic chains.

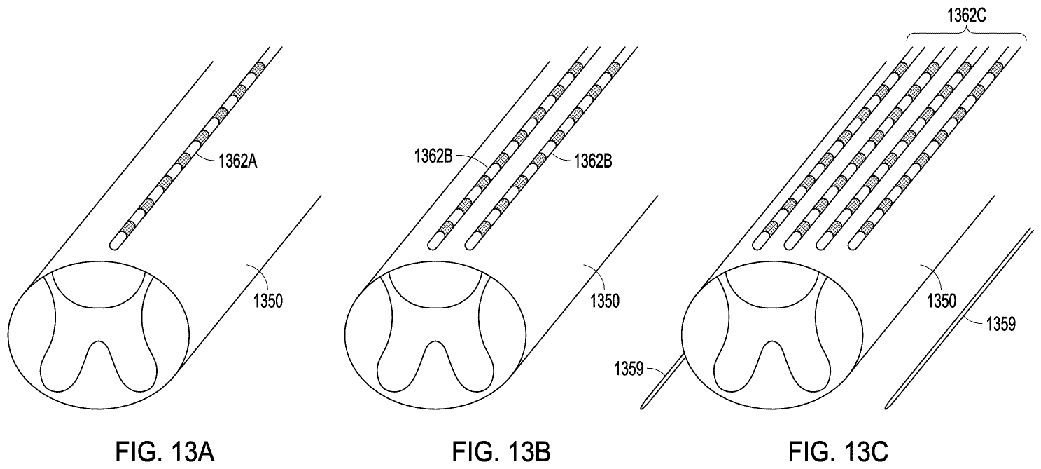

FIGS. 13A-13C illustrate, by way of example and not limitation, various electrode arrangements that may be used to stimulate various spinal tracts within the spinal cord. FIG. 13A is a schematic view of a single electrical modulation lead 1362A implanted over approximately the longitudinal midline of the patient's spinal cord 1350. Such placement may be useful to capture nerve tracts on the dorsal side of the spinal cord such as the gracile fasciculus tract and the cuneate fasciculus tract. The single lead may be moved laterally off of the midline to be closer to a targeted nerve tract. It is understood that additional leads or lead paddle(s) may be used, such as may be used to provide a wider electrode arrangement, to provide the electrodes closer to various nerve tracts, and to fractionalize energy contributions across the leads to provide a focused neuromodulation field on at least a portion of one or more nerve tracts. For example, FIG. 13B is a schematic view of two electrical modulation leads 1362B implanted in the dorsal epidural space, and FIG. 13C is a schematic view of four electrical modulation leads 1362C implanted in the dorsal epidural space. Some embodiments may position one or more lead(s) outside of the dorsal epidural space (e.g., in lateral or ventral epidural space and/or outside of the epidural space). The additional electrodes provide greater ability to focus a neuromodulation field to modulate one or more targeted nerve tracts or a targeted portion(s) of one or more nerve tracts. Any other plurality of leads or a multiple column paddle lead can also be used.

A ring electrode allows current to project equally in every direction from the position of the electrode, and typically does not enable stimulus current to be directed from only a particular angular position or a limited angular range around the lead. A lead which includes only ring electrodes may be referred to as a non-directional lead. However, the electrodes may include one or more ring electrodes, and/or one or more sets of segmented electrodes (or any other combination of electrodes). A lead which includes at least one or more segmented electrodes may be referred to as a directional lead. Some embodiments may non-directional or directional leads to stimulate the sympathetic chains 1359.

Segmented electrodes may provide better current steering than ring electrodes. Through the use of a radially segmented electrode array, current steering may be performed not only along a length of the lead but also around a circumference of the lead. This may provide precise three-dimensional targeting and delivery of the current stimulus to neural target tissue, while potentially avoiding stimulation of other tissue. In some examples, segmented electrodes can be together with ring electrodes. In an example, there can be different numbers of segmented electrodes at different longitudinal positions. Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes. The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead and/or around the circumference of the lead.

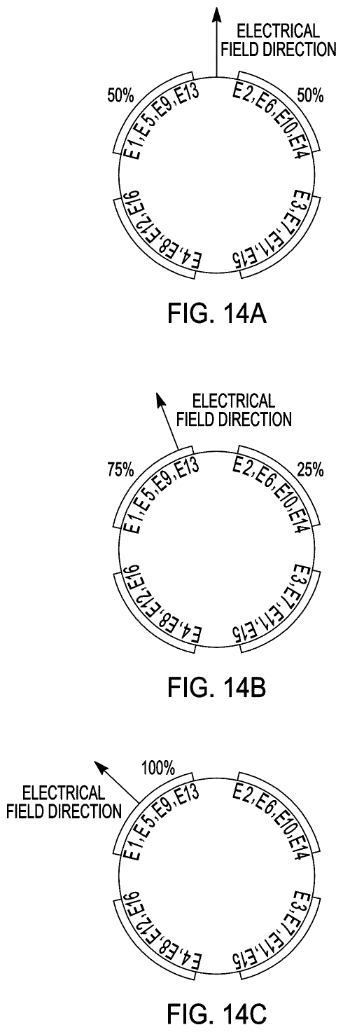

FIGS. 14A-14C illustrate examples of directional leads with segmented electrodes that are circumferentially and axially disposed about the leads. By way of non-limiting examples, each neuromodulation lead may carry sixteen electrodes, arranged as four rings of electrodes (the first ring consisting of electrodes E1-E4; the second ring consisting of electrodes E5-E8; the third ring consisting of electrodes E9-E12; and the fourth ring consisting of electrodes E13-E16) or four axial columns of electrodes (the first column consisting of electrodes E1, E5, E9, and E13; the second column consisting of electrodes E2, E6, E10, and E14; the third column consisting of electrodes E3, E7, E11, and E15; and the fourth column consisting of electrodes E4, E8, E12, and E16). The actual number and shape of leads and electrodes may vary according to the intended application. To generate electrical fields in different medio-lateral directions, the electrodes may have different current fractionalizations in the radial direction. To generate electrical fields in different rostro-caudal directions, the electrodes may have different current fractionalizations in the longitudinal direction.

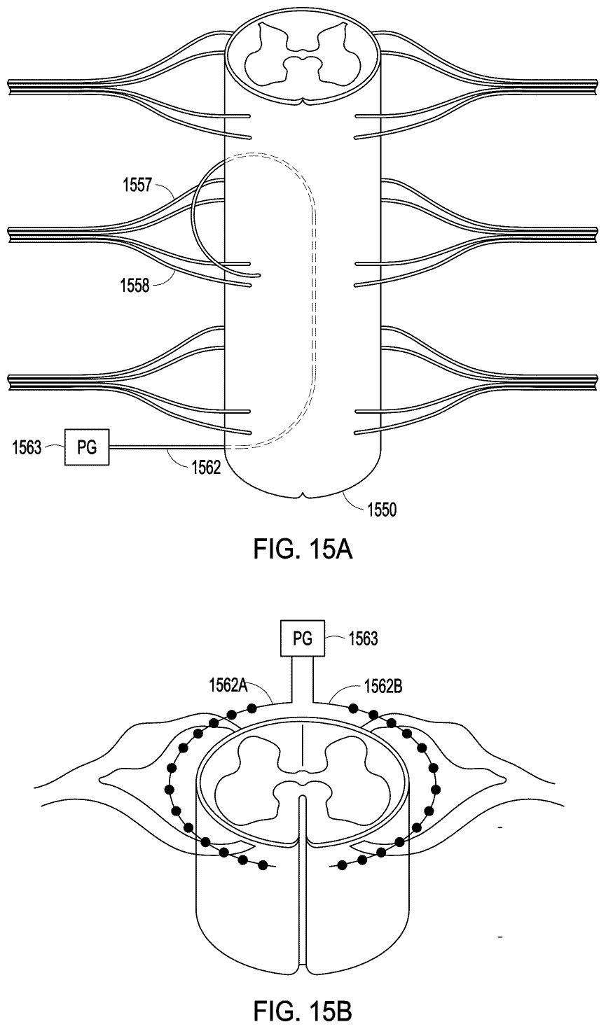

Some embodiments may implant a portion of the lead(s) along the lateral side(s) of the spinal cord to better target some of the nerve tracts, such as one or more of the tracts along the lateral side of the spinal cord as illustrated in FIG. 11. FIG. 15A illustrates a portion of the spinal cord 1550, with nerve roots 1557, 1558 extending from three vertebral locations, and further illustrates a neural stimulation lead 1562 fed through the dorsal epidural space (behind the illustrated cord) and least partially around the spinal cord 1550 to operationally set electrodes in place to stimulate activity in at least some nerve pathways on the lateral side of the spinal cord. At least some of the electrodes may be in a lateral or antero-lateral space surrounding the spinal cord. The neuromodulator 1563 can be implanted in an appropriate location, such as in an abdominal region or in or just above the buttocks.

FIG. 15B illustrates a multi-lead embodiment to stimulate neural targets on contralateral sides of the spinal cord. The illustrated figure shows two leads 1562A, 1562B exiting from a neurostimulator. One lead is directed around a first side, and a second lead is directed around a second side. Electrodes, illustrated as filled-in circles, on each lead are placed operationally in position with respect to the neural targets to stimulate the neural target(s) and elicit the desired effect(s). Some embodiments may deliver neuromodulation to inhibit nerve activity in some neural elements such as to inhibit nerve activity in the nerve roots or in untargeted nerve tracts in the spinal cord.

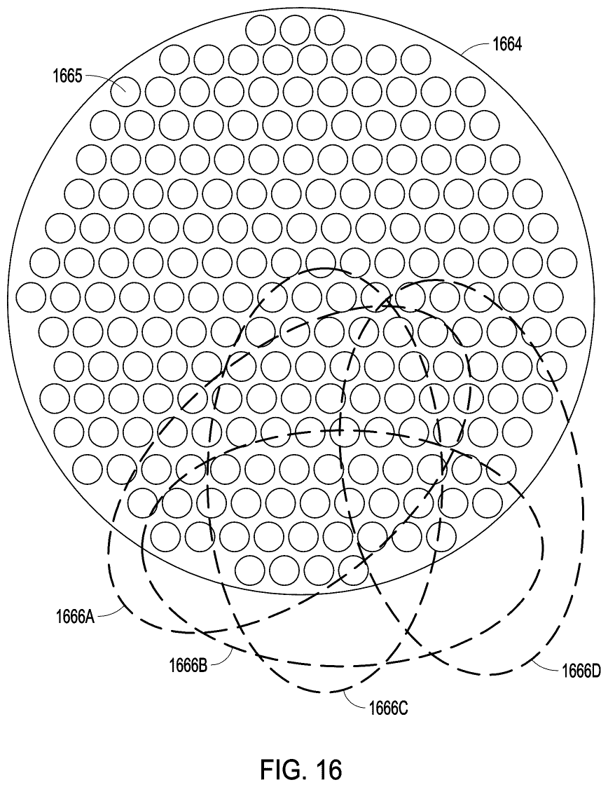

FIG. 16 illustrates a nerve tract 1664 with a plurality of pathways (not to scale) 1665 and neuromodulation field(s) (e.g., 1666A, 1666B, 1666C, 1666D) focused to modulate some of the plurality of pathways in the nerve tract. With multiple electrodes proximate to the targeted neural pathways, the system can choose active electrodes and fractionalize the current contributions of these active electrodes to provide a modulation field with a tighter focus. The neurostimulation system may be configured to use different electrode configurations (e.g., different active electrodes and different fractionalized energy contributions for the active electrodes). For example, the figure illustrates four modulation fields (e.g., 1666A, 1666B, 1666C, 1666D), where each field corresponds to a different electrode configuration.

The neurostimulation system may be configured to deliver different electrical fields to achieve a temporal summation of modulation in nerve pathways in one or more of the nerve tracts. Some embodiments may deliver the fields at the same time (e.g., either starting and stopping at the same time or overlapping in time). For example, four independent stimulation channels may be used to generate the illustrated four fields. Some embodiments rotate the four fields. Thus, for example, one stimulation channel may be used to rotate through the four fields. Multiple stimulation channels may be used to rotate through the fields. The electrical fields can be generated respectively on a pulse-by-pulse basis. For example, a first electrical field can be generated by the electrodes (using a first current fractionalization) during a first electrical pulse of the pulsed waveform, a second different electrical field can be generated by the electrodes (using a second different current fractionalization) during a second electrical pulse of the pulsed waveform, a third different electrical field can be generated by the electrodes (using a third different current fractionalization) during a third electrical pulse of the pulsed waveform, a fourth different electrical field can be generated by the electrodes (using a fourth different current fractionalized) during a fourth electrical pulse of the pulsed waveform, and so forth. Similarly, rather than one pulse at a time, the system may use bursts of pulses to generate each of the first, second, third and fourth electrical fields. These electrical fields may be rotated or cycled through multiple times under a timing scheme, where each field is implemented using a timing channel. The electrical fields may be generated at a continuous pulse rate or may be bursted on and off. Furthermore, the interpulse interval (i.e., the time between adjacent pulses), pulse amplitude, and pulse duration during the electrical field cycles may be uniform or may vary within the electrical field cycle. That is, the modulation waveform may be tonic with regular repeating pulses with constant stimulation parameters or may be irregular patterns with varied stimulation parameters.

The field(s) may be focused to modulate some but not all of the axons withing a tract. Rather, some embodiments may target axons that project to a specific part of the thalamus that will transmit impulses to a specific brain region (without targeting other axons that project to other parts of the thalamus). This may be beneficial to more specifically target regions of the brain for epilepsy without causing unnecessarily impulses to other parts of the brain.

Selective neural stimulation may be delivered using stimulation patterns (see Spix et al., “Population-Specific Neuromodulation Prolongs Therapeutic Benefits of Deep Brain Stimulation.” Science 374, 201-206 (2021); Gittis et al, 2014, “New Roles for the External Globus Pallidus in Basal Ganglia Circuits and Behavior.” The Journal of Neuroscience, Nov. 12, 2014⋅34(46):15178-15183; Mastro et al., “Cell-Specific Pallidal Intervention Induces Long-Lasting Motor Recovery in Dopamine Depleted Mice.” Nat Neurosci. 2017 June; 20(6): 815-823. doi:10.1038/nn.4559, each of which is incorporated by reference in its entirety). Such stimulation patterns may allow a specific subpopulation of cells to be activated without activating other subpopulations. Additionally, stimulation patterns may be used that prevent habituation. (See Paschen et al., “Long-Term Efficacy of Deep Brain Stimulation for Essential Tremor: an Observer-Blinded Study.” Neurology. 2019 Mar. 19; 92(12):e1378-e1386. doi: 10.1212/WNL.0000000000007134. Epub 2019 Feb. 20. PMID: 30787161; Seir, et al., “Alternating Thalamic Deep Brain Stimulation for Essential Tremor: A Trial to Reduce Habituation.”. Movement Disorders Clinical Practice 2018; 5(6): 620-626. doi: 10.1002/mdc3.12685; Petry-Shmelzer, et al., “Daily Alternation of DBS Settings Does Not Prevent Habituation of Tremor Suppression in Essential Tremor Patients.” Movement Disorders Clinical Practice 2019; 6(5): 417-418. doi: 10.1002/mdc3.12777, each of which is incorporated by reference in its entirety). Furthermore, stimulation patterns may be used to increase the efficiency of the stimulation (See Brocker et al., “Optimized Temporal Pattern of Brain Stimulation Designed by Computational Evolution.” Sci. Transl. Med. 9, eaah3532 (2017), incorporated by reference in its entirety). For example, fewer pulses may be delivered using a pattern. The overall efficacy may be maintained using the pattern, but less energy is used since fewer pulses are delivered. This is beneficial to reduce battery consumption. Stimulation patterns may be developed to induce plasticity (See Huh, Yul, et al. “Combination Of 1 kHz And 60 Hz Spinal Cord Stimulation Inhibits Mechanical And Cold Hypersensitivity With Prolonged Wash-Out In A Rat Spared Nerve Injury Model Of Neuropathic Pain.” The Journal of Pain 24.4 (2023): 56; Pfeiffer et al. “Coordinated Reset Vibrotactile Stimulation Induces Sustained Cumulative Benefits in Parkinson's Disease. Front. Physiol. 12:624317. doi: 10.3389/fphys.2021.624317; and Lo et al. “Paired Electrical Pulse Trains for Controlling Connectivity in Emotion-Related Brain Circuitry.” DOI 10.1109/TNSRE.2020.3030714, IEEE Transactions on Neural Systems and Rehabilitation Engineering, each of which is incorporated by reference in its entirety). Plasticity implies that stimulation benefits outlast the stimulation. Thus, benefits of stimulation may persist for a period of time after turning off the stimulation. Inducing plasticity remodels (e.g., rewires) neural connections. Huh, Yul, et al.

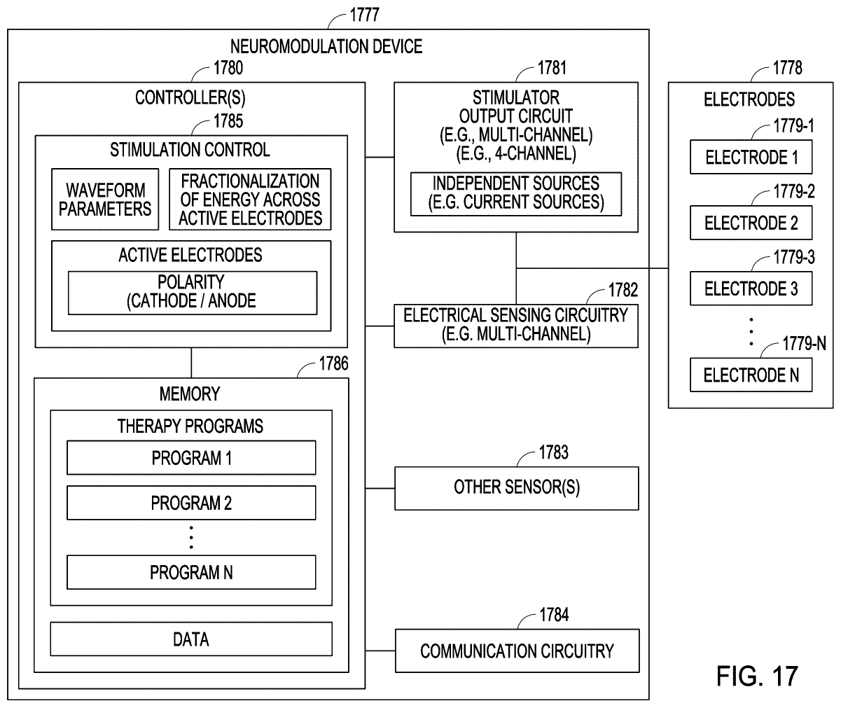

FIG. 17 illustrates, by way of example and not limitation, a neuromodulation device with a plurality of different programs for delivering neuromodulation. The neuromodulation device 1777 may correspond to the implantable waveform generator and/or the ETM in FIG. 1, for example. The neuromodulation device 1777 may be connected to a lead system 1778 which includes one or more leads each configured to be electrically connected to the neuromodulation device 1777 and a plurality of electrodes 1779-1 to 1779-N distributed in an electrode arrangement using the one or more leads. The illustrated neuromodulation device 1777 includes one or more controllers 1780 operably connected to a stimulator output circuit 1781 to deliver neuromodulation to the electrodes. The stimulator output circuit 1781 may include a plurality of independent sources such as independent current sources for each electrode. The stimulator output circuit 1781 may be configured as a multi-channel (such as but not limited to four channels) system capable of simultaneously and independently generating and delivering separate stimulation waveforms to different electrode combinations. Some embodiments of the neuromodulation device 1777 may include electrical sensing circuitry 1782 configured to sense electrical activity (e.g., local field potentials, evoked compound actions potentials, evoked resonant neural activity (ERNA), electrospinogram, or other electrical signals) using at least some of the electrodes. Some embodiments of the neuromodulation device 1777 may include other sensor(s) 1783 that may be used to control the neuromodulation or provide context for the therapy or other events, or to detect events. Some embodiments of the neuromodulation device 1777 may include communication circuitry 1784 used to communicate with at least one external device. The controller(s) 1780 may be configured to provide stimulation control 1785 to control the neuromodulation generated by the stimulator output circuit 1781 and delivered to the electrodes, which may include the waveform parameters for the neuromodulation, the active electrodes and polarity of the active electrodes used to deliver the neuromodulation, and the fractionalization of energy across the active electrodes. The controller(s) 1780 may include memory 1786 configured to store data and configured to store therapy programs. The memory 1786 of the illustrated neuromodulation device 1777 may include programs. The device 854 may be configured with a plurality of stimulation channels, and one or more programs may be available for selection for each of the channel(s). Two or more different programs may be configured to stimulate different neurostimulation targets such as but not limited to stimulating different tracts in the spinal cord or different axons within a tract. For example, a first neuromodulation field (e.g., from a first program) may be configured to stimulate a first site and a second neuromodulation field (e.g., from a second program) may be configured to stimulate a second site lateral to the first site, the first neuromodulation field may be configured to stimulate a first nerve tract without stimulating a second nerve tract, and the second neuromodulation field may be configured to stimulate the second nerve tract without stimulating the first nerve tract; or the first neuromodulation field is at a first spinal cord level without extending to a second spinal cord level, and the second neuromodulation field is at the second spinal cord level without extending to the first spinal cord level.

Two or more programs may stimulate the same neurostimulation target using different stimulation parameters such as but not limited to different pulse parameters (e.g., pulse amplitude, pulse frequency, pulse width), different therapy schedules, different patterns of pulses, different waveforms and the like). The stored data in the memory 1786 may include a variety of data used to deliver the therapy, to evaluate the delivered therapy, and/or to determine when events are detected and corresponding therapies to provide in response to the detection of the events. By way of example and not limitation, the data may include sensor data (such as but not limited to data from heart rhythm, heart rate, pulse and blood flow, muscle activity, respiration, activity, posture or electrical activity such as ecaps, local field potentials or evoked resonant neural activity (ERNA), motion detectors, camera images, microphone recordings) or operational data (e.g., impedance, battery charge, etc.) for the device.

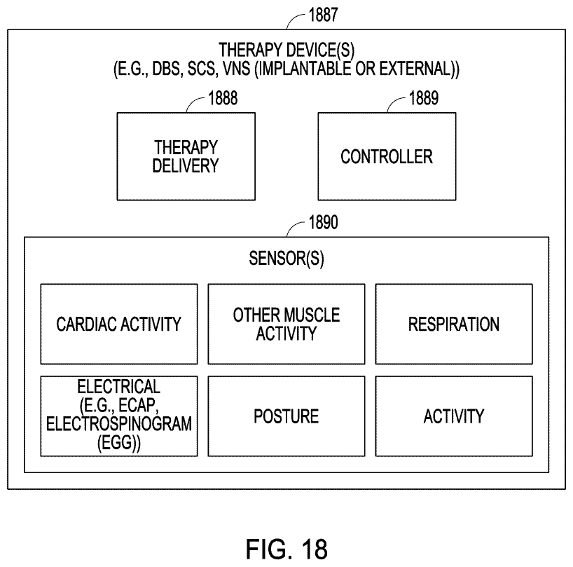

FIG. 18 illustrates, by way of example and not limitation, a therapy device with a sensing function. The illustrated therapy device 1887 may include a therapy delivery circuit 1888 (e.g., waveform generator) and a controller 1889 configured to control the therapy delivery circuit to output the desired waveform. The therapy device 1887 may also include different sensors 1890 that may be used to provide feedback for therapy, to monitor a condition of the patient undergoing the therapy, or to trigger the start or stop of the therapy. By way of example and not limitation, the therapy device(s) includes (include) sensors configured to detect cardiac activity such as but not limited to heart rhythm, heart rate, pulse and blood flow, muscle activity, respiration such as but not limited to respiration rate and volume, activity, posture or electrical activity such as ecaps, local field potentials or evoked resonant neural activity (ERNA). The sensor(s) of the therapy device(s) may be used to detect events, such as epileptic events, to appropriately respond to the events or to determine the efficacy of the stimulation in treating the epileptic events.

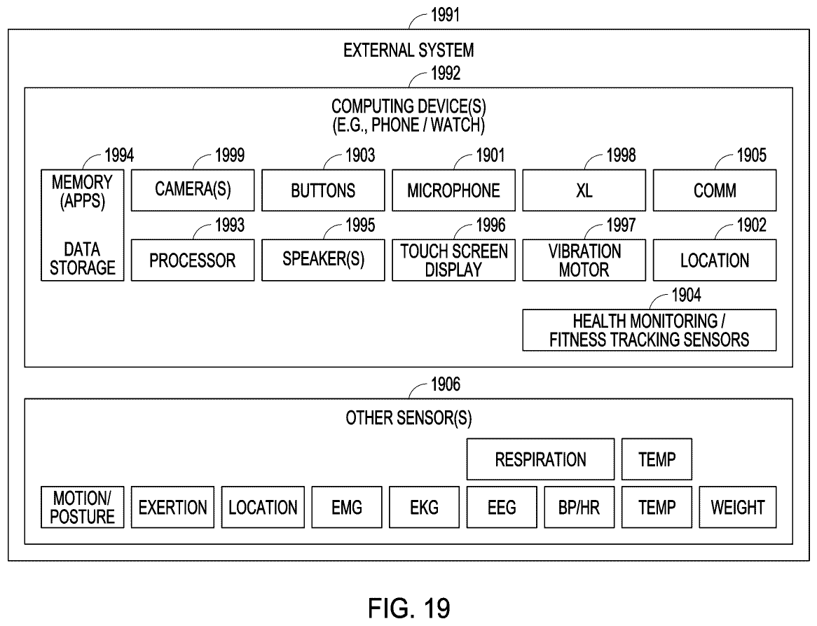

FIG. 19 illustrates, by way of example and not limitation, an external system with sensing function. The external system 1991 may include one or more computing devices 1992. The computing device(s) 1992 may include a phone, a tablet, and/or a wearable such as a watch, by way of example. The computing device(s) may include a number of features that may be used, alone or in combination with other features, by the system to detect events and/or respond to detected events. For example, the computing device(s) (1992 e.g., phone, watch, and the like) may include a processor 1993 and a memory 1994 including apps to be implemented by the processor to perform various processes. The memory 1994 may provide data storage for the external system. Other features of the computing device(s) 1992 may include at least one of speaker(s) 1995 to produce acoustic signals, a touch screen display 1996 for providing a user interface used to receive user inputs and provide visual outputs, and a vibration motor 1997 that may be used to provide a haptic output. The computing device(s) 1992 may include feature(s) capable of being used alone or in various combinations to detect an event. For example, accelerometer(s) (XL) 1998, gyroscope, camera(s) 1999, microphone(s) 1901 and/or location service(s) 1902 may be used, with or without a clock and information about the patient's activities at different time(s) and/or location(s), to determine events. The location service(s) may use global positioning system (GPS), Wi-Fi and cellular towers (e.g., triangulation of Wi-Fi access points and/or cellular towers), Bluetooth beacons or Radio Frequency Identification (RFID). A determined location may be used to detect an event. For example, XLs 1180 can detect motion and/or posture. Microphones 1901 can be used to detect patient effort (breathing, grunts) and sound at the patient's location that may be used to detect an event. Cameras 1999 may be used to detect motion, location, and patient effort, such as via a shaky image, facial feature(s) of the patient, visual detection of an epileptic event, bradykinesia event or other event. The touch screen display 1996 may be used to determine attempted use of the computing device (e.g., phone) via an unlocked screen or other interaction with the device via the touch screen display or other button(s) 1903 on the device, which may be used to detect an event. The computing device(s) 1992 may include health monitoring/fitness tracking sensor(s) 1904 that may make use of other sensor(s) in the device. Health monitoring/fitness tracking sensor(s) 1904 may be configured to detect at least an estimate of heart rate, ECG, blood pressure, oxygen, steps, sleep, exercise, stress, and the like. A more exhaustive list of sensor(s) of computing device(s) may include any one or various combinations of an accelerometer, a gyroscope, a magnetometer/compass, a barometric pressure sensor, a body temperature sensor, a heart rate monitor, an oximetry sensor, an ambient light sensor, a bioimpedance sensor, a proximity sensor, an orientation sensor, a pedometer, a calorie counter, an ECG sensor, a gesture sensor, a UV sensor, an electrodermal activity sensor, a skin conductance sensor, and a GPS sensor.

The computing device(s) 1992 in the external system 1991 also includes a number of features that may be used to detect events and/or respond to a detected event. For example, the computing device(s) 1992 may include communication technology 1905 (e.g., Wi-Fi, Bluetooth) for use to communicate with other computing device(s), other sensor(s), and/or other perceptible signal transducer(s). Other sensor(s) 1906 may include other motion, exertion and/or posture sensors, other exertion sensor(s), other sensor(s) for detecting location (e.g., beacon, such as within range of a Bluetooth device), other sensors of physiological parameters such as EEG, EMG, EKG, respiration, galvanic skin response (GSR), cardiovascular parameters such blood pressure, rhythm and/or heart rate, temperature, and weight.

The present subject matter may be used in an open loop mode. For example, a prophylactic therapy to prevent or ameliorate epileptic seizures may be delivered. The target(s) for the therapy, stimulation pattern and stimulation timing may be programmed into the system. By way of example this programming may be determined based programming session with the patient and/or based on a model.

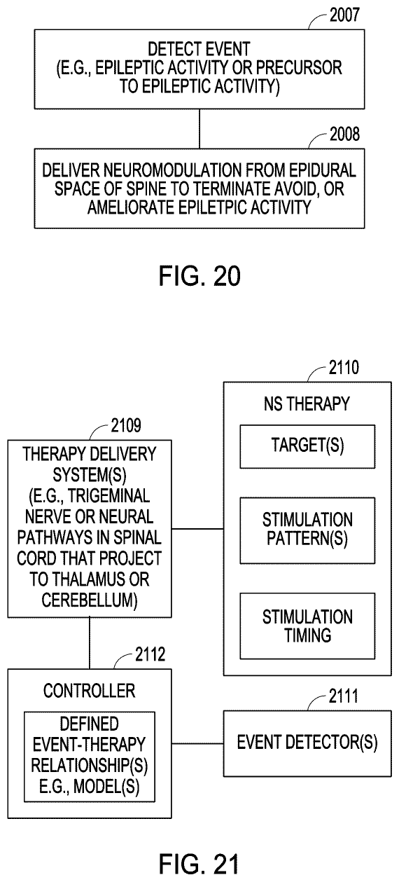

Some embodiments (open or closed loop) may be configured to receive input(s) to detect an event associated epilepsy such as epileptic activity or precursor(s) to epileptic activity. FIG. 20 illustrates, by way of example and not limitation, a method for delivering an epileptic therapy in response to a detected event. At 2007, the system may be configured to detect an event such as epileptic activity or a precursor to epileptic activity. For example, as seizures are electrical activity that change sensations, behaviors, awareness, and movements, some embodiments may use one or more indicators of electrical activity in the brain and/or changes in behavior, awareness and/or movement as inputs used to detect a seizure, stages of a seizure, or precursors to a seizure. Other inputs may relate to a patient state when the event occurs as the desired therapy may be different when the patient is sleeping or resting than if the patient is active. At 2008, the system responds to the detected event by delivering the epileptic therapy. Some embodiments deliver neuromodulation therapy using electrode(s) positioned in the epidural space to terminate, avoid or ameliorate the epileptic activity.

FIG. 21 illustrates, by way of example and not limitation, a system for treating epilepsy using at least one event detector. The system may include at least one therapy delivery system 2109 configured to deliver one or more therapies 2110. Different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies). The therapy targets may include targets identified in FIG. 6 or may include other therapy targets. For example, an SCS system may be used to deliver one or more therapies to one or more of the neural targets identified in FIG. 6 (e.g., trigeminal nerve or neural pathways in or near the spinal cord that transmit impulses to the thalamus or cerebellum). The therapy delivery system 2109 is configured to coordinate the therapy(ies). The system may include at least one event detector 2111 configured to detect at least one predefined event. The event(s) may be determined to be relevant to the epileptic condition being treated by the therapy delivery system or otherwise relevant to the efficacy of the delivered therapy.

For example, some embodiments may use one or more indicators of electrical activity in the brain and/or muscles, and/or heart rate, accelerometers, cameras, microphones and the like to detect seizures, stages of a seizure or precursors to a seizure. Some embodiments may receive from a user device inputs that relate to a patient state or condition (e.g., mood, activity, function, sleep, and the like). This information may be used to identify an event to trigger the therapy. By way of example and not limitation, the event detector(s) may be configured to detect predefined epileptic event(s) using at least one of: received user input; sensed electrical signals in a brain; sensed cardiac activity; sensed blood oxygen; sensed respiration; sensed movement or lack of movement; sensed body temperature; sensed neurotransmitter or biochemical component; sensed sound, sensed ultrasound, or analysis of an image of a patient. This information may also be used to evaluate the therapeutic effect of the therapy. The system may include a controller 2112 configured to control the therapy delivery system(s) based on a defined event-therapy relationship (e.g., model). The model(s) may be used to determine the appropriate therapy for these inputs. For example, various models may be developed and used to determine the appropriate therapy(ies) that should be delivered in response to the event(s) to treat the epileptic patient. The controller 2112 may be implemented in therapy-delivery system(s) 2109 or may be one or more separate controllers (e.g., programmer(s), remote control(s), phone(s), tablet(s) and the like) configured to communicate and with the therapy-delivery device(s) in the therapy delivery system 2109. The event-therapy relationships may function to map certain inputs to an appropriate therapeutic output (e.g., focused inducement of impulses in an area of the brain) to provide therapeutic stimulation that also avoids stimulating certain regions that would have an undesirable effect (e.g., “side effect”) on the patient.

The system may be configured to respond to more than one event with different neural stimulation targets to treat the therapy, different patterns, and/or different timing. Thus, for example, the system can treat different types of focal seizures, a generalized seizure, or a focal seizure that develops into a generalized seizure. For example, the therapy may be configured to interrupt a progression of seizure activity.

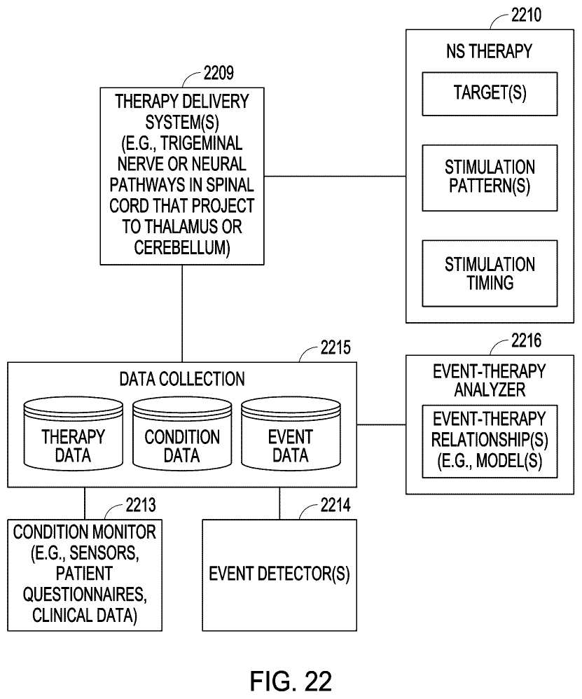

FIG. 22 illustrates, by way of example and not limitation, a system configured for defining event-therapy relationship(s) that associates therapies to detected events. Similar to FIG. 21, the system includes at least one therapy delivery system 2209 configured to deliver one or more therapies 2210 to one or more therapy targets. Different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies), The system may include a condition monitor(s) 2213 to detect a patient condition that is the condition being treated by the therapies or a condition associated with the patient condition being treated or to the therapies being delivered to the patient (including side effects, comorbidities, medication/medication schedule, and the like). The system may also include at least one event detector(s) 2214 to detect events. Machine learning (or other artificial intelligence) may be implemented to identify event(s) that appear to have an effect the patient or the efficacy of the epilepsy therapy(ies) delivered to the patient. The system may include a data collection system 2215 to detect therapy data (e.g., therapy configuration data such as stimulation parameters, neural stimulation site(s), stimulation patterns, stimulation timing, and the like) for the therapy(ies), condition data from the condition monitor, and event data from the event detector(s). The event-therapy analyzer 2216 may be configured to use machine learning (or other artificial intelligence) to analyze the collected data, event data and condition data to identify event-therapy relationship(s) (e.g., develop models) that may be used by the controller in the system of FIG. 21. A plurality of events may be detected to compile event data. The event data, the therapy data, and the condition data may be analyzed to determine whether one or more of the therapies are effective in treating the condition when delivered in response to one or more of the detected plurality of events and define one or more event-therapy relationships associating the one or more of the therapies to be delivered in response to the detected event(s). The event-therapy analyzer 2216 may operate to update a model by training, re-training, or updating a model (e.g., an artificial intelligence model, such as a neural network) based on the analyzed data. One or multiple instances of a model may be trained to generate programs and program parameters, for any one or more of two or more therapies. The models may be patient-specific or may be developed for a larger population.

A system may use machine learning or other artificial intelligence to identify stimulation pattern(s) for neural target(s). These stimulation patterns may be developed to enhance selective neural stimulation of the neural target(s) without modulating non-targeted cells, to prevent habituation to neural signal, to increase the efficiency of the stimulation and/or increase efficacy of the stimulation, and to induce plasticity.

As identified above, machine learning may be used to identify event-therapy relationships, to identify the events that have an effect on the patient's condition or on the therapy being delivered to the patient and/or to identify stimulation pattern(s) for neural target(s) to achieve desired results such as enhanced selective neural stimulation, avoidance of habituation, increased efficiency, increased efficacy and/or induced plasticity. Machine-learning programs (MLPs), also referred to as machine-learning algorithms or tools, are utilized to perform operations associated with machine learning tasks, such as identifying relationship(s) in the collected data, including feature(s) in a sensed signal, different neurostimulation therapies, and waveform parameter(s) used to control the different neurostimulation therapies. Machine learning is a field of study that gives computers the ability to learn without being explicitly programmed. Machine learning explores the study and construction of algorithms that may learn from existing data (e.g., “training data”) and make predictions about new data. Such machine-learning tools may build a model from example training data in order to make data-driven predictions or decisions expressed as outputs or assessments. The machine-learning algorithms use the training data to find correlations among identified features that affect the outcome. The machine-learning algorithms use features for analyzing the data to generate assessments. A feature is an individual measurable property of the observed phenomenon. In the context of a biological signal, some examples of features may include, but are not limited to, peak(s) such as a minimum peak, a maximum peak as well as local minimum and maximum peaks, a range between peaks, a difference in values for features, a feature change with respect to a baseline, an area under a curve, a curve length, an oscillation frequency, and a rate of decay for peak amplitude. Inflection points in the signal may also be an observable feature of the signal, as an inflection point is a point where the signal changes concavity (e.g., from concave up to concave down, or vice versa), and may be identified by determining where the second derivative of the signal is zero. Detected feature(s) may be partially defined by time (e.g., length of curve over a time duration, area under a curve over a time duration, maximum or minimum peak within a time duration, etc.). The machine-learning algorithms use the training data to find correlations among the identified features that affect the outcome or assessment. With the training data and the identified features, the machine-learning tool is trained. The machine-learning tool appraises the value of the features as they correlate to the training data. The result of the training is the trained machine-learning program. Various machine learning techniques may be used to train models to make predictions based on data fed into the models. During a learning phase, the models are developed against a training dataset of inputs to optimize the models to correctly predict the output for a given input. A training data set may be defined for desired functionality of the closed-loop algorithm and closed loop parameters may be defined for desired functionality of the closed-loop algorithm. Generally, the learning phase may be supervised, semi-supervised, or unsupervised; indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset. Models may be run against a training dataset for several epochs (e.g., iterations), in which the training dataset is repeatedly fed into the model to refine its results. For example, in a supervised learning phase, a model is developed to predict the output for a given set of inputs and is evaluated over several epochs to more reliably provide the output that is specified as corresponding to the given input for the greatest number of inputs for the training dataset. In another example, for an unsupervised learning phase, a model is developed to cluster the dataset into groups and is evaluated over several epochs as to how consistently it places a given input into a given group and how reliably it produces the n desired clusters across each epoch.

Once an epoch is run, the models are evaluated, and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several machine learning algorithms that may be applied with the present disclosure, including linear regression, random forests, decision tree learning, neural networks, deep neural networks, and the like. New data is provided as an input to the trained machine-learning program, and the trained machine-learning program generates the assessment as output. The assessment that is output may be out of an expected range (e.g., anomalous), indicating that remedial action such as retraining of the machine learning algorithm(s) is warranted. The system also may be configured to determine that the new data includes anomalous data with respect to the training data that was used to train the machine-learning program. The detection of new data that is anomalous may trigger remedial action(s) such as, if it is determined that the previously used training data is outdated, retraining the machine learning program using updated training data.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method for treating a patient with epileptic seizures, comprising:
using a set of electrodes epidurally positioned adjacent to a spinal cord or dorsal column nuclei and a stimulation configuration to deliver neuromodulation energy to at least one neural target in or near the spinal cord, wherein:
the neural target includes the dorsal column nuclei or axons projecting to the dorsal column nuclei,
the dorsal column nuclei and the axons are somatotopically organized and the dorsal column nuclei project to at least one of another brainstem area, a cerebellum or a thalamus, and the at least one of the another brainstem area, the cerebellum or the thalamus includes somatotopically organized nuclei and project axons to different areas of at least one of a cortex or a limbic system, and
the at least one neural target corresponds to at least one of the cortex, the limbic system, the another brainstem area, the cerebellum or the thalamus in which the epileptic seizures propagate.

2. The method of claim 1, wherein the at least one neural target includes a spinothalamic tract that provides afferent fibers to an intralaminar thalamic nuclei.

3. The method of claim 2, wherein the at least one neural target is based on a symptom location where the patient experiences a symptom caused by the epileptic seizures.

4. The method of claim 1, wherein the epileptic seizures include a first symptom at a first location and a second symptom at a second location, and a first target is based on the first location where the patient experiences the first symptom, and a second target is based on the second location where the patient experiences the second symptom.

5. The method of claim 1, wherein the stimulation configuration includes a plurality of pulses in a pulse pattern, wherein the pulse pattern includes at least one of different pulse amplitudes, different pulse widths, different pulse-to-pulse spacing, different pulse shapes, different bursts of pulses, different electrode contacts in the set of electrodes, or different fractionalizations.

6. The method of claim 1, wherein the epileptic seizures propagate to two or more areas, and the method includes using two or more stimulation configurations corresponding to the two or more areas, wherein the two or more stimulation configurations include different stimulation patterns, and at least one of the different stimulation patterns includes a stimulation pattern with different pulse amplitudes, different pulse widths, or different pulse-to-pulse spacing.

7. The method of claim 1, wherein the at least one neural target includes axons in at least a portion of a gracile fasciculus or a cuneate fasciculus.

8. The method of claim 1, wherein the at least one neural target includes pathways in at least a portion of a posterolateral tract, lateral spinothalamic tract, an anterior spinothalamatic tract, a spinoreticular tract, a posterior spinocerebellar tract, an anterior spinocerebellar tract, or a spinoolivary tract.

9. The method of claim 1, wherein the at least one neural target includes pathways in at least a portion of trigeminothalamatic tract via a trigeminocervical nucleus.

10. The method of claim 1, including using at least one lead in a dorsal epidural space to position at least some of the set of electrodes in the dorsal epidural space.

11. The method of claim 1, including using at least one lead fed through a dorsal epidural space and at least partially around the spinal cord to position at least some of the set of electrodes in a lateral or antero-lateral epidural space surrounding the spinal cord.

12. The method of claim 1, wherein the stimulation configuration is configured to focus a neuromodulation field to stimulate neural pathways in a portion of a nerve tract without stimulating other neural pathways in other portions of the nerve tract.

13. The method of claim 12, including:
using a first channel and a first neuromodulation field to deliver first neuromodulation energy to provide a first neuromodulation field and using a second channel and a second stimulation configuration to deliver second neuromodulation energy to provide a second neuromodulation field; and
coordinating delivery of the first neuromodulation energy and the second neuromodulation energy, wherein
the first neuromodulation field is configured to stimulate a first site, and the second neuromodulation field is configured to stimulate a second site lateral to the first site;
the first neuromodulation field is configured to stimulate a first nerve tract without stimulating a second nerve tract, and the second neuromodulation field is configured to stimulate the second nerve tract without stimulating the first nerve tract; or
the first neuromodulation field is at a first spinal cord level without extending to a second spinal cord level, and the second neuromodulation field is at the second spinal cord level without extending to the first spinal cord level.

14. The method of claim 1, further comprising using at least one event detector to detect at least one predefined epileptic event, and responding to the detected at least one predefined epileptic event by delivering the neuromodulation energy.

15. The method of claim 14, wherein the at least one event detector is configured to detect at least a first predefined epileptic event and a second predefined epileptic event; and the method includes responding to the first predefined epileptic event by delivering a first neuromodulation therapy and responding to the second predefined epileptic event by delivering a second neuromodulation therapy, wherein the first and second neuromodulation therapies differ in at least one of a neural target or use different neural stimulation patterns.

16. A method, comprising:
delivering at least one epilepsy therapy to at least one therapy target in or near a spinal cord using a set of electrodes to provide therapy data, wherein the therapy data includes therapy configuration data and wherein the at least one therapy target is used to transmit impulses to at least one of a cerebellum, a thalamus, a brainstem or a limbic system;
detecting one or more epileptic events to compile event data;
providing condition data indicative of an effect that the delivered at least one therapy has on a treated condition;
analyzing the event data, the therapy configuration data, and the condition data to:
determine whether the at least one therapy is effective in treating the one or more epileptic events when delivered in response to the detected one or more epileptic events; and
define one or more event-therapy relationships associating the at least one epilepsy therapy to be delivered in response to the one or more detected epileptic events.

17. A system for treating a patient with epileptic seizures, comprising:
a therapy delivery system configured to use a set of electrodes positioned adjacent to a spinal cord or dorsal column nuclei and a stimulation configuration to deliver neuromodulation energy to at least one neural target in or near the spinal cord, wherein:
the at least one neural target includes the dorsal column nuclei or axons projecting to the dorsal column nuclei,
the dorsal column nuclei and the axons are somatotopically organized and the dorsal column nuclei project to at least one of another brainstem area, a cerebellum or a thalamus, and the at least one of the another brainstem area, the cerebellum or the thalamus includes somatotopically organized nuclei and project axons to different areas of at least one of a cortex or a limbic system, and
the at least one neural target corresponds to at least one of the cortex, the limbic system, the another brainstem area, the cerebellum or the thalamus in which the epileptic seizures propagate.

18. The system according to any of claim 17, wherein the epileptic seizures include a first symptom at a first location and a second symptom at a second location, and a first target is based on the first location where the patient experiences the first symptom and a second target is based on the second location where the patient experiences the second symptom.

19. The system according to any of claim 17, wherein the therapy delivery system is configured to:
use a first channel to deliver first neuromodulation energy using a first stimulation configuration to provide a first neuromodulation field and use a second channel to deliver second neuromodulation energy using a second stimulation configuration to provide a second neuromodulation field; and
coordinate delivery of the first neuromodulation energy and the second neuromodulation energy, wherein
the first neuromodulation field is configured to stimulate a first site, and the second neuromodulation field is configured to stimulate a second site lateral to the first site;
the first neuromodulation field is configured to stimulate a first nerve tract without stimulating a second nerve tract, and the second neuromodulation field is configured to stimulate the second nerve tract without stimulating the first nerve tract; or
the first neuromodulation field is at a first spinal cord level without extending to a second spinal cord level, and the second neuromodulation field is at the second spinal cord level without extending to the first spinal cord level.

20. The system according to any of claim 17, further comprising at least one event detector to detect at least one predefined epileptic event, wherein the therapy delivery system is configured to respond to the detected at least one predefined epileptic event by delivering the neuromodulation energy, wherein the at least one event detector is configured to detect the at least one predefined epileptic event using at least one of: received user input; sensed electrical signals in a brain; sensed cardiac activity; sensed blood oxygen; sensed respiration; sensed movement or lack of movement; sensed body temperature; sensed neurotransmitter or biochemical component; sensed sound, sensed ultrasound, or analysis of an image of a patient, wherein the at least one event detector is configured to detect at least a first predefined epileptic event and a second predefined epileptic event; and the therapy delivery system is configured to respond to the first predefined epileptic event by delivering a first neuromodulation therapy and respond to the second predefined epileptic event by delivering a second neuromodulation therapy, wherein the first and second neuromodulation therapies differ in at least one of a neural target or use different neural stimulation patterns.
